SEQUENCE LISTING (12 sequences)

SEQ ID NO: 1 - DNA, 2256 bp, Homo sapiens
  source 1..2256: mol_type = genomic DNA
ctgggtcccgactgcccacctcctcctcctccccctccccccaacaacaacaacaacaacaactccaagcacaccggccataagagtgcgtgtgtccccaacatgaccgaacgaagaagggacgagctctctgaagagatcaacaacttaagagagaaggtcatgaagcagtcggaggagaacaacaacctgcagagccaggtgcagaagctcacagaggagaacaccacccttcgagagcaagtggaacccacccctgaggatgaggatgatgacatcgagctccgcggtgctgcagcagctgctgccccaccccctccaatagaggaagagtgcccagaagacctcccagagaagttcgatggcaacccagacatgctggctcctttcatggcccagtgccagatcttcatggaaaagagcaccagggatttctcagttgatcgtgtccgtgtctgcttcgtgacaagcatgatgaccggccgtgctgcccgttgggcctcagcaaagctggagcgctcccactacctgatgcacaactacccagctttcatgatggaaatgaagcatgtctttgaagaccctcagaggcgagaggttgccaaacgcaagatcagacgcctgcgccaaggcatggggtctgtcatcgactactccaatgctttccagatgattgcccaggacctggattggaacgagcctgcgctgattgaccagtaccacgagggcctcagcgaccacattcaggaggagctctcccacctcgaggtcgccaagtcgctgtctgctctgattgggcagtgcattcacattgagagaaggctggccagggctgctgcagctcgcaagccacgctcgccaccccgggcgctggtgttgcctcacattgcaagccaccaccaggtagatccaaccgagccggtgggaggtgcccgcatgcgcctgacgcaggaagaaaaagaaagacgcagaaagctgaacctgtgcctctactgtggaacaggaggtcactacgctgacaattgtcctgccaaggcctcaaagtcttcgccggcgggaaaactccccggccccgctgtagagggaccttcagcgaccgggccagaaataataaggtccccacaagatgatgcctcatctccacacttgcaagtgatgcttcagatacatctgccaggcaggcacacactcttcgtacgagctatgattgattcaggggcatctggtaacttcatagatcacgagtatgtagctcagaacggaattcctttgagaataaaggactggcccatcttggttgaagctatcgatgggaggcctatagcttccggtcccgtggtgcatgaaacacatgatcttattgtcgatcttggggatcaccgcgaagtcctctcatttgacgtaacccagtcccctttcttccccgtggtcctcggagtgcggtggctgagtactcacgatcctaatattacatggagcacccggtcaatcgtatttgatagtgagtactgtaggtaccactgtcgaatgtatagtcctattccccctagtttgcctcccccagctcctcaaccacccctgtactatcccgtggacggatatagggtgtaccagcccgttcgatattactacgtacaaaatgtttatacacctgttgatgagcacgtctacccagaccatagactcgtcgaccctcacatcgaaatgattccaggagcccatagtataccttcagggcacgtatactctctgtccgaacctgaaatggcagctcttcgagattttgtggcaagaaatgtaaaagatgggctaattactccaacgattgcacctaatggagcccaagttctccaggtgaagagggggtggaaactgcaagtttcttatgattgccgagctccaaacaattttactatccagaatcagtatcctcgcctatctattccaaatttagaagaccaagcacacctggcaacgtacactgaattcgtacctcaaatacctggataccaaacataccccacatatgccgcgtacccgacctacccagtaggattcgcctggtacccagtgggacgagacggacaaggaagatcactatatgtacctgtgatgatcacttggaatccacactggtaccgccagcctccggtaccacagtacccgccgccacagccgccgcctccaccaccaccaccgccgccgcctccatcttacagtaccctgtatccgtatgatgttccggattatgcatga

SEQ ID NO: 2 - DNA, 1536 bp, Vesiculovirus indiana
  source 1..1536: mol_type = genomic DNA
ttactttccaagtcggttcatctctatgtctgtataaatctgtcttttcttggtgtgctttaatttaatgcaaagatggataccaactcggagaaccaagaatagtccaatgattaaccctatgataaagaaaaaagaggcaatagagcttttccaactactgaaccaaccttctacaagctcgattggatttttggatagcccagtatcaccaaaaaataaactctcatcatcaggaagttgcgaagcagcgtcttgaatgtgaggatgttcgaacacctgagcctttgagctaagatgaagatcggagtccaacataccatgtccaatcatgtataaaggaaacttatatcctgaactggtcctcagaactccattgggtccaatttccacgtcttcatatggtgcccagtcatcccacagttccctttctgtggtagttccactgatcattccgaccattcttgagaggattggagcagcaatatcgactctgatgtatctggtctcaaagtattttagggtaccattgattatggtgaaagcaggaccggttcctgggtttttaggagcaagatagctgagatccactggagagattggaagacccgctctgattttgctccaggtttcttggcagagggaataatccaagatcctctcaacgtcctgaattagacttacatccactgaggtctgagatggagcagagatacttgacccttctgggcattcagggaatctggctgcagcaaagagatccttatcagccatctcgaaccagacacctgatgggagtctgactccccaatgcttgcagtattgcattttgcaggccttgcctccagtttcataagcaaagtagttacttctgaaccctgtgccctcctttcccagggatgatagctctccgtcctctgagaagaaggtgatgtccatggaaatgaggttagaatcacatagccctttgaccttatagtcagaatgccaggttgtagagttatggacagtggggcatatgtaattgctgcattttccgttgatgaactgtgaatcaacccattctcctgtgtattcatcaaccagcacatggtgaggagtcacctggacaatcactgcttcggcatccgtcacagttgcatatccacaactttgaggagggaagcctggattcagccaagttccttgtttcgtttgttcaatgctttccttgcattgttctacagatggagtgaaggatcggatggaatgtgttatatacttcggtccataccagcggaaatcacaagtagtgacccatttggaagcatgacacatccaaccgtctgcttgaatagccttgtgactcttgggcattttgacttgtaaggctgtgcctattaagtcattatgccaatttaaatctgagcttgacgggcaataatggtaattagaaggaacatttttccagtttcctttttggttgtgtggaaaaactatggtgaacttgcaattcaccccaatgaataaaaaggctaagtacaaaaggcacttcat

SEQ ID NO: 3 - DNA, 2361 bp, synthetic construct
  source 1..2361: mol_type = other DNA
atgaagtgccttttgtacttagcctttttattcattggggtgaattgccaccatcaccatcaccaccaggtgcagctggtgcagagcggcgcggaagtgaaaaaaccgggcgcgagcgtgaaagtgagctgcaaagcgagcggctatacctttaccggctattatatgcattgggtgcgccaggccccgggccagggcctggaatggatgggccgcattaacccgaacagcggcggcaccaactatgcgcagaagtttcagggccgcgtgaccatgacccgcgataccagcattagcaccgcgtatatggaactgagccgcctgcgcagcgatgataccgccgtgtattattgcgcccgcgataaaggcgcgggccgcattaccatggtgcgcggcgtgaaacgcaactatggcatggatgtgtggggccagggcaccaccgtgaccgtgagcagcggcggcggcggcagcggcggcggcggcagcggcggcggcggcagcgatgtggtgatgacccagagcccgctgagcctgccggtgaccctgggccagcccgcgagcattagctgccgcagcagccagaacattgtgcatagcgatggcaacacctatctgaactggtttcagcagcgccccggccagagcccgcgccgcctgatttataaagtgagcaaccgcgatagcggcgtgccggatcgctttagcggcagcggcagcggcaccgattttaccctgaaaattagccgcgtggaagcggaagatgtgggcgtgtattattgctttcagggcacccattggccgtttacctttggccagggcacccgcctggaaattggtggaggctctggcggtggctcctctggtggtggaagcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaa

SEQ ID NO: 4 - DNA, 840 bp, synthetic construct
  source 1..840: mol_type = other DNA
gtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaagcttagccatggcttcccgccggaggtggaggagcaggatgatggcacgctgcccatgtcttgtgcccaggagagcgggatggaccgtcaccctgcagcctgtgcttctgctaggatcaatgtg

SEQ ID NO: 5 - DNA, 1773 bp, Homo sapiens
  source 1..1773: mol_type = genomic DNA
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatccggcggcggcgggagtggcggcgggggttctggcggaggcctcgctagcggtggctccatggtcagcgttccaacagcctcaccctcggcatccagcagctcctctcagtgccggtccagcatgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctga

SEQ ID NO: 6 - DNA, 741 bp, Homo sapiens
  source 1..741: mol_type = genomic DNA
atctcaagctccccagccaaggaccctcctatacagcgtttgcgcggcgcagtaacacggtgtgaggatggtcaactgtttatatcatcttacaaaaatgaatatcagactatggaagtgcaaaataactcagtagtaattaagtgcgacggcctgtatatcatttatctgaagggaagtttcttccaggaggtcaagatcgaccttcatttccgagaggaccacaatccaatctctatcccaatgttgaatgacggtcgacgtatcgtgtttacagttgtggcaagcctcgcattcaaggataaggtttatcttaccgtaaatgcacctgataccctctgcgaacacttgcaaattaatgatggagaattgatagtagtgcaattgactccaggttattgtgctcctgaaggcagttatcattccaccgtaaaccaagtgcccctcgcggccgctggcagccctgggccacctggtcccccaggtcctccaggacccccagggcccccaggcccccccgggccgcctggacccccagggccaccaggccccccaggcccttcctctggcggaaccggtggatccctggtgccgcgcggcagccctggaggctcctggagccacccgcagttcgaaaagcgtcgaggagggccagaacaaaaactcatctcagaagaggatctgaatagcgccgtcgacggcagcagccatcatcaccatcaccatagccgcggactcgag

SEQ ID NO: 7 - DNA, 768 bp, Homo sapiens
  source 1..768: mol_type = genomic DNA
caggtgcagctggtgcagagcggcgcggaagtgaaaaaaccgggcgcgagcgtgaaagtgagctgcaaagcgagcggctatacctttaccggctattatatgcattgggtgcgccaggccccgggccagggcctggaatggatgggccgcattaacccgaacagcggcggcaccaactatgcgcagaagtttcagggccgcgtgaccatgacccgcgataccagcattagcaccgcgtatatggaactgagccgcctgcgcagcgatgataccgccgtgtattattgcgcccgcgataaaggcgcgggccgcattaccatggtgcgcggcgtgaaacgcaactatggcatggatgtgtggggccagggcaccaccgtgaccgtgagcagcggcggcggcggcagcggcggcggcggcagcggcggcggcggcagcgatgtggtgatgacccagagcccgctgagcctgccggtgaccctgggccagcccgcgagcattagctgccgcagcagccagaacattgtgcatagcgatggcaacacctatctgaactggtttcagcagcgccccggccagagcccgcgccgcctgatttataaagtgagcaaccgcgatagcggcgtgccggatcgctttagcggcagcggcagcggcaccgattttaccctgaaaattagccgcgtggaagcggaagatgtgggcgtgtattattgctttcagggcacccattggccgtttacctttggccagggcacccgcctggaaatt

SEQ ID NO: 8 - protein, 751 aa, Homo sapiens
LGPDCPPPPPPPPPNNNNNNNSKHTGHKSACVPNMTERRRDELSEEINNLREKVMKQSEENNNLQSQVQKLTEENTTLREQVEPTPEDEDDDIELRGAAAAAAPPPPIEEECPEDLPEKFDGNPDMLAPFMAQCQIFMEKSTRDFSVDRVRVCFVTSMMTGRAARWASAKLERSHYLMHNYPAFMMEMKHVFEDPQRREVAKRKIRRLRQGMGSVIDYSNAFQMIAQDLDWNEPALIDQYHEGLSDHIQEELSHLEVAKSLSALIGQCIHIERRLARAAAARKPRSPPRALVLPHIASHHQVDPTEPVGGARMRLTQEEKERRRKLNLCLYCGTGGHYADNCPAKASKSSPAGKLPGPAVEGPSATGPEIIRSPQDDASSPHLQVMLQIHLPGRHTLFVRAMIDSGASGNFIDHEYVAQNGIPLRIKDWPILVEAIDGRPIASGPVVHETHDLIVDLGDHREVLSFDVTQSPFFPVVLGVRWLSTHDPNITWSTRSIVFDSEYCRYHCRMYSPIPPSLPPPAPQPPLYYPVDGYRVYQPVRYYYVQNVYTPVDEHVYPDHRLVDPHIEMIPGAHSIPSGHVYSLSEPEMAALRDFVARNVKDGLITPTIAPNGAQVLQVKRGWKLQVSYDCRAPNNFTIQNQYPRLSIPNLEDQAHLATYTEFVPQIPGYQTYPTYAAYPTYPVGFAWYPVGRDGQGRSLYVPVMITWNPHWYRQPPVPQYPPPQPPPPPPPPPPPPSYSTLYPYDVPDYA

SEQ ID NO: 9 - protein, 511 aa, Vesiculovirus indiana
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 10 - protein, 786 aa, synthetic construct
MKCLLYLAFLFIGVNCHHHHHHQVQLVQSGAEVKKPGASVKVSCKASGYTFTGYYMHWVRQAPGQGLEWMGRINPNSGGTNYAQKFQGRVTMTRDTSISTAYMELSRLRSDDTAVYYCARDKGAGRITMVRGVKRNYGMDVWGQGTTVTVSSGGGGSGGGGSGGGGSDVVMTQSPLSLPVTLGQPASISCRSSQNIVHSDGNTYLNWFQQRPGQSPRRLIYKVSNRDSGVPDRFSGSGSGTDFTLKISRVEAEDVGVYYCFQGTHWPFTFGQGTRLEIGGGSGGGSSGGGSKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 11 - protein, 256 aa, Homo sapiens
QVQLVQSGAEVKKPGASVKVSCKASGYTFTGYYMHWVRQAPGQGLEWMGRINPNSGGTNYAQKFQGRVTMTRDTSISTAYMELSRLRSDDTAVYYCARDKGAGRITMVRGVKRNYGMDVWGQGTTVTVSSGGGGSGGGGSGGGGSDVVMTQSPLSLPVTLGQPASISCRSSQNIVHSDGNTYLNWFQQRPGQSPRRLIYKVSNRDSGVPDRFSGSGSGTDFTLKISRVEAEDVGVYYCFQGTHWPFTFGQGTRLEI

SEQ ID NO: 12 - DNA, 2580 bp, synthetic construct
  source 1..2580: mol_type = other DNA
atctcaagctccccagccaaggaccctcctatacagcgtttgcgcggcgcagtaacacggtgtgaggatggtcaactgtttatatcatcttacaaaaatgaatatcagactatggaagtgcaaaataactcagtagtaattaagtgcgacggcctgtatatcatttatctgaagggaagtttcttccaggaggtcaagatcgaccttcatttccgagaggaccacaatccaatctctatcccaatgttgaatgacggtcgacgtatcgtgtttacagttgtggcaagcctcgcattcaaggataaggtttatcttaccgtaaatgcacctgataccctctgcgaacacttgcaaattaatgatggagaattgatagtagtgcaattgactccaggttattgtgctcctgaaggcagttatcattccaccgtaaaccaagtgcccctcgcggccgctggcagccctgggccacctggtcccccaggtcctccaggacccccagggcccccaggcccccccgggccgcctggacccccagggccaccaggccccccaggcccttcctctggcggaaccggtggatccctggtgccgcgcggcagccctggaggctcctggagccacccgcagttcgaaaagcgtcgaggagggccagaacaaaaactcatctcagaagaggatctgaatagcgccgtcgacggcagcagccatcatcaccatcaccatagccgcggactcgagggaagcggagctactaacttcagcctgctgaagcaggctggagacgtggaggagaaccctggacctatgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatccggcggcggcgggagtggcggcgggggttctggcggaggcctcgctagcggtggctccatggtcagcgttccaacagcctcaccctcggcatccagcagctcctctcagtgccggtccagcatgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctga

(19) United States
(12) Patent Application Publication
(10) Pub. No.: US 2025/0375393 A1
(43) Pub. Date: Dec. 11, 2025

(54) PROTEIN-BASED NANOPARTICLE FOR SELF-PACKAGING AND DELIVERING MRNA, PREPARATION METHOD THEREOF AND PHARMACEUTICAL COMPOSITION

(71) Applicant: NATIONAL TSING HUA UNIVERSITY, HSINCHU (TW)

(72) Inventors: Yu-Chen HU, HSINCHU (TW); Pin-Yan CHEN, HSINCHU (TW); Ting-Lun TIEN, HSINCHU (TW)

(21) Appl. No.: 18/769,432

(22) Filed: Jul. 11, 2024

(30) Foreign Application Priority Data
Jun. 5, 2024 (TW) 113120805

(51) Int. Cl. A61K 9/51; A61K 48/00; A61P 35/00; A61P 37/04; C07K 14/005; C07K 16/28; C12N 5/071; C12N 7/00; C12N 15/86

(52) U.S. Cl. CPC A61K 9/5184; A61K 9/5192; A61K 48/005; A61P 35/00; A61P 37/04; C07K 14/005; C07K 16/2863; C12N 5/0686; C12N 7/00; C12N 15/86; C07K 2317/622; C12N 2710/14022; C12N 2710/14043; C12N 2710/14051; C12N 2760/20222

(57) ABSTRACT

A method for preparing protein-based nanoparticle for self-packaging and delivering mRNA includes the following steps. A first donor plasmid, a second donor plasmid and a third donor plasmid are provided. A plasmid transposing step is performed. A recombinant virus preparing step is performed so as to obtain a first recombinant baculovirus, a second recombinant baculovirus and a third recombinant baculovirus. A transducing step is performed, wherein the first recombinant baculovirus, the second recombinant baculovirus and the third recombinant baculovirus are used to infect a producer cell so as to express a nucleocapsid protein, an envelope protein, an engineered envelope protein and a target RNA, and the nucleocapsid protein, the envelope protein, the engineered envelope protein and the target RNA are self-assembled to form a protein-based nanoparticle for self-packaging and delivering mRNA.

### RELATED APPLICATIONS

This application claims priority to Taiwan Application Serial Number 113120805, filed Jun. 5, 2024, which is herein incorporated by reference.

### SEQUENCE LISTING XML

A sequence listing XML submitted as an xml file via Patent Center is incorporated herein by reference. The sequence listing XML file submitted via Patent Center with the name “CP-6589-US_SEQ_LIST” was created on Jul. 11, 2024, which is 26,645 bytes in size.

### BACKGROUND

### Technical Field

The present disclosure relates to a nanoparticle, a preparation method thereof and a pharmaceutical composition. More particularly, the present disclosure relates to a protein-based nanoparticle for self-packaging and delivering mRNA, a preparation method thereof and a pharmaceutical composition including the aforementioned protein-based nanoparticle for self-packaging and delivering mRNA.

### Description of Related Art

The pseudo-virus nanoparticle (“PVNP” hereafter) is a particle whose structure is similar to the structure of a natural virus. The PVNP has a nucleocapsid protein of the natural virus but does not have a virus genome, and thus there is no doubt that the PVNP does not infect a cell or replicate in the cell. Therefore, the PVNP can be used as a safe nucleic acid delivery vector and widely applied in clinical applications such as vaccine development, gene treatment, etc.

Currently, the PVNP is prepared by transfecting the plasmids with a nucleocapsid protein gene, an envelope protein gene and a target gene into a producer cell so as to facilitate the producer cell to express a nucleocapsid protein, an envelope protein and a target RNA. The nucleocapsid protein in the producer cell can recognize a packaging signal from the target RNA and spontaneously assemble with the target RNA to form the PVNP, and then the surface of the PVNP can have the envelope protein by the budding mechanism.

However, the method of transfecting the plasmids into the producer cell to prepare the PVNP is costly, and the production efficiency thereof is not satisfactory. Further, the transfection efficiency of the conventional PVNP is not ideal, and the conventional PVNP lacks the cell-targeting property, which in turn reduces the treatment effect of the PVNP.

Therefore, how to develop a method that can enhance the production efficiency of the self-assembling nanoparticle and reduce the production cost of the self-assembling nanoparticle so as to obtain the self-assembling nanoparticle prepared therefrom to have excellent transfection efficiency and cell-targeting property, has become a subject in the related fields of academia and industry.

### SUMMARY

According to one aspect of the present disclosure, a method for preparing protein-based nanoparticle for self-packaging and delivering mRNA includes the following steps. A first donor plasmid, a second donor plasmid and a third donor plasmid are provided, wherein the first donor plasmid includes a nucleocapsid protein gene and an envelope protein gene, the second donor plasmid includes an engineered envelope protein gene, and the third donor plasmid includes a target gene. A plasmid transposing step is performed, wherein the first donor plasmid is transposed to a first shuttle vector, the second donor plasmid is transposed to a second shuttle vector, and the third donor plasmid is transposed to a third shuttle vector, wherein the first shuttle vector is transfected to a first virus-amplifying cell, the second shuttle vector is transfected to a second virus-amplifying cell, and the third shuttle vector is transfected to a third virus-amplifying cell. A recombinant virus preparing step is performed, wherein the first virus-amplifying cell, the second virus-amplifying cell and the third virus-amplifying cell are cultured so as to obtain a first recombinant baculovirus, a second recombinant baculovirus and a third recombinant baculovirus, wherein the first recombinant baculovirus is obtained from the first virus-amplifying cell, the second recombinant baculovirus is obtained from the second virus-amplifying cell, and the third recombinant baculovirus is obtained from the third virus-amplifying cell. A transducing step is performed, wherein the first recombinant baculovirus, the second recombinant baculovirus and the third recombinant baculovirus are used to infect a producer cell so as to express a nucleocapsid protein, an envelope protein, an engineered envelope protein and a target RNA, and the nucleocapsid protein, the envelope protein, the engineered envelope protein and the target RNA are self-assembled so as to form a protein-based nanoparticle, and the protein-based nanoparticle is for self-packaging and delivering mRNA. The protein-based nanoparticle includes a lipid carrier, the nucleocapsid protein, the envelope protein, the engineered envelope protein and the target RNA, the target RNA and the nucleocapsid protein are located in the lipid carrier, the target RNA is covered by the nucleocapsid protein, and the envelope protein and the engineered envelope protein are separately located on a surface of the lipid carrier.

According to another aspect of the present disclosure, a protein-based nanoparticle is provided. The protein-based nanoparticle is prepared by the method for preparing protein-based nanoparticle for self-packaging and delivering mRNA according to the aforementioned aspect.

According to further another aspect of the present disclosure, a pharmaceutical composition includes the protein-based nanoparticle according to the aforementioned aspect. The pharmaceutical composition is for treating a cancer.

### BRIEF DESCRIPTION OF THE DRAWINGS

The present disclosure can be more fully understood by reading the following detailed description of the embodiment, with reference made to the accompanying drawings as follows:

FIG. 1 is a flow chart of a method for preparing protein-based nanoparticle for self-packaging and delivering mRNA according to one embodiment of the present disclosure.

FIG. 2 is a schematic view of gene construction of a first donor plasmid, a second donor plasmid and a third donor plasmid used in the method for preparing protein-based nanoparticle for self-packaging and delivering mRNA of FIG. 1.

FIG. 3 is a schematic view of a protein-based nanoparticle of the present disclosure.

FIG. 4 shows the results of the functional titer of a nanoparticle of Comparative example 1 and a protein-based nanoparticle of Example 1.

FIG. 5 shows the results of Western blot analysis of a cell lysate of Untransduced group, a cell lysate of Transduced group and the protein-based nanoparticle of Example 1.

FIG. 6A shows the results of the functional titer of protein-based nanoparticles of Example 2 to Example 4 and a nanoparticle of Comparative example 2.

FIG. 6B shows the results of the transfection efficiency of the protein-based nanoparticles of Example 2 to Example 4 and the nanoparticle of Comparative example 2 used to transfect CT26 cells.

FIG. 7 shows the results of the transfection efficiency of the protein-based nanoparticles of Example 2 to Example 4 and the nanoparticles of Comparative example 2 used to transfect different types of cells.

FIG. 8 is a schematic view of genes carried by a first recombinant baculovirus, a second recombinant baculovirus and a third recombinant baculovirus in Example 5.

FIG. 9 shows the results of concentration of IL-12 at different time points after the CT26 cells are transfected with different doses of protein-based nanoparticles of Example 5.

FIG. 10A shows the results of concentration of IL-12 at different time points after the CT26 cells are respectively transfected with a nanoparticle of Comparative example 3 and the protein-based nanoparticle of Example 5.

FIG. 10B shows the results of concentration of OX40L at different time points after the CT26 cells are respectively transfected with the nanoparticle of Comparative example 3 and the protein-based nanoparticle of Example 5.

FIG. 11A shows the results of concentration of IFN-γ at different time points after the CT26 cells are respectively transfected with the nanoparticle of Comparative example 3 and the protein-based nanoparticle of Example 5.

FIG. 11B shows the results of concentration of TNF-α at different time points after the CT26 cells are respectively transfected with the nanoparticle of Comparative example 3 and the protein-based nanoparticle of Example 5.

FIG. 12A shows the results of the proportion of CD4⁺ T cells expressing CD69 after the CT26 cells are respectively transfected with the nanoparticle of Comparative example 3 and the protein-based nanoparticle of Example 5.

FIG. 12B shows the results of the proportion of CD8⁺ T cells expressing CD69 after the CT26 cells are respectively transfected with the nanoparticle of Comparative example 3 and the protein-based nanoparticle of Example 5.

FIG. 13A shows the tumor-growing curves of mouse tumor models of Control group 2 within 30 days.

FIG. 13B shows the tumor-growing curves of mouse tumor models of Comparison group 2 within 30 days.

FIG. 13C shows the tumor-growing curves of mouse tumor models of Example 6 within 30 days.

FIG. 13D shows the tumor-growing curves of mouse tumor models of Example 7 within 30 days.

FIG. 14 shows the survival rates of the mouse tumor models of Control group 2, Comparison group 2, Example 6 and Example 7 within 30 days.

FIG. 15A shows the results of concentration of IFN-γ in serum at 24 hours and 72 hours after the mouse tumor models of Control group 2, Comparison group 2, Example 6 and Example 7 are administrated with respective treatment dosages.

FIG. 15B shows the results of concentration of TNF-α in serum at 24 hours and 72 hours after the mouse tumor models of Control group 2, Comparison group 2, Example 6 and Example 7 are administrated with respective treatment dosages.

FIG. 16A shows the quantitative results of CD4⁺ T cells in tumor samples of the mouse tumor models of Control group 2, Comparison group 2, Example 6 and Example 7 on Day 15 of the experiment.

FIG. 16B shows the quantitative results of CD8⁺ T cells in the tumor samples of the mouse tumor models of Control group 2, Comparison group 2, Example 6 and Example 7 on Day 15 of the experiment.

FIG. 17A shows the quantitative results of CD4⁺ T cells in spleen samples of the mouse tumor models of Control group 2, Comparison group 2, Example 6 and Example 7 on Day 15 of the experiment.

FIG. 17B shows the quantitative results of CD8⁺ T cells in the spleen samples of the mouse tumor models of Control group 2, Comparison group 2, Example 6 and Example 7 on Day 15 of the experiment.

FIG. 18 shows the results of the proportion of M2 macrophages of the mouse tumor models of Control group 2, Comparison group 2, Example 6 and Example 7 on Day 15 of the experiment.

### DETAILED DESCRIPTION

The present disclosure will be further exemplified by the following specific embodiments. However, the embodiments can be applied to various inventive concepts and can be embodied in various specific ranges. The specific embodiments are only for the purposes of description and are not limited to these practical details thereof.

### Method for Preparing Protein-Based Nanoparticle for Self-Packaging and Delivering mRNA of the Present Disclosure

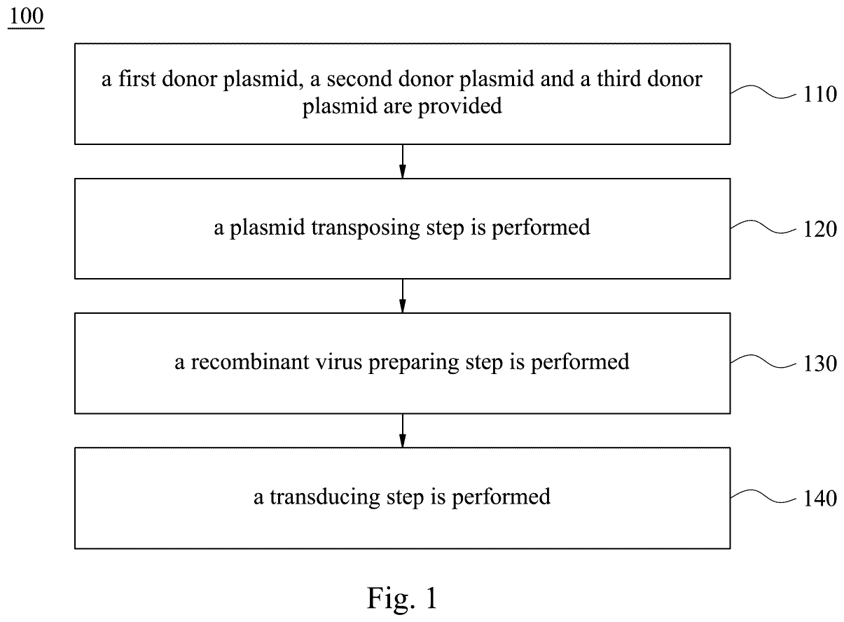

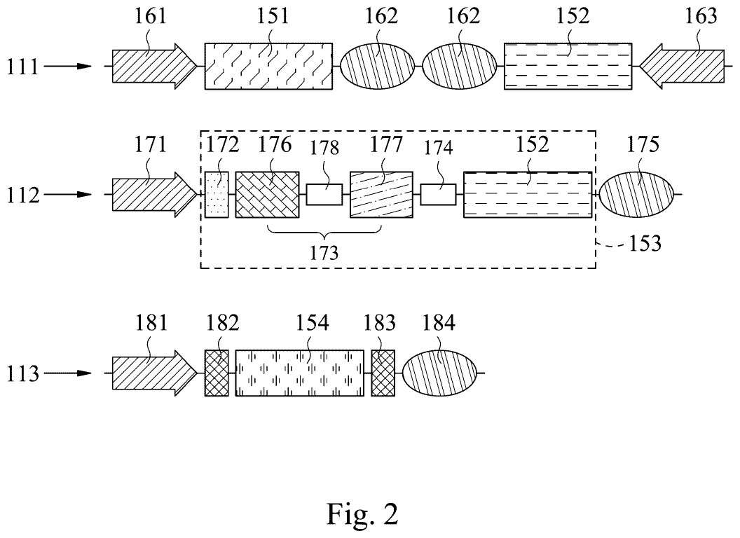

Reference is made to FIG. 1 and FIG. 2. FIG. 1 is a flow chart of a method 100 for preparing protein-based nanoparticle for self-packaging and delivering mRNA according to one embodiment of the present disclosure. FIG. 2 is a schematic view of gene construction of a first donor plasmid 111, a second donor plasmid 112 and a third donor plasmid 113 used in the method 100 for preparing protein-based nanoparticle for self-packaging and delivering mRNA of FIG. 1. The method 100 for preparing protein-based nanoparticle for self-packaging and delivering mRNA includes Step 110, Step 120, Step 130 and Step 140.

In Step 110, the first donor plasmid 111, the second donor plasmid 112 and the third donor plasmid 113 are provided, wherein the first donor plasmid 111 includes a nucleocapsid protein gene 151 and an envelope protein gene 152, the second donor plasmid 112 includes an engineered envelope protein gene 153, and the third donor plasmid 113 includes a target gene 154. In detail, as shown in FIG. 2, the first donor plasmid 111 can sequentially include a first promoter 161, the nucleocapsid protein gene 151, two poly-A-tails 162, the envelope protein gene 152 and a second promoter 163, wherein the transcription direction of the first promoter 161 is different from that of the second promoter 163. Hence, the expression of the nucleocapsid protein gene 151 and the expression of the envelope protein gene 152 can be simultaneously driven by the first promoter 161 and the second promoter 163. The second donor plasmid 112 can sequentially include a third promoter 171, a signal peptide gene 172, a single chain variable fragment gene 173, a linker gene 174, the envelope protein gene 152 and a poly-A-tail 175, wherein the single chain variable fragment gene 173 includes a heavy chain variable region 176 and a light chain variable region 177, the heavy chain variable region 176 is joined to the light chain variable region 177 by a linker gene 178, and the engineered envelope protein gene 153 is composed of the signal peptide gene 172, the single chain variable fragment gene 173, the linker gene 174 and the envelope protein gene 152. The third donor plasmid 113 can sequentially include a fourth promoter 181, an untranslated region 182, the target gene 154, an untranslated region 183 and a poly-A-tail 184. Further, the first promoter 161, the second promoter 163, the third promoter 171 and the fourth promoter 181 can be the promoters of cytomegalovirus (“CMV” hereafter) so as to facilitate the gene expressions of the nucleocapsid protein gene 151, the envelope protein gene 152, the engineered envelope protein gene 153 and the target gene 154 in high level. Further, the linker gene 174 and the linker gene 178 can be GS linker genes, but the present disclosure is not limited thereto.

Furthermore, the nucleocapsid protein gene 151 and the envelope protein gene 152 are integrated into the Tn7 site of a pFastBac-dual plasmid by a Gibson assembly method so as to obtain the first donor plasmid 111, and the engineered envelope protein gene 153 and the target gene 154 are respectively integrated into the Tn7 sites of the other two pFastBac-dual plasmids by the Gibson assembly method so as to obtain the second donor plasmid 112 and the third donor plasmid 113.

Moreover, in FIG. 2, the nucleocapsid protein gene 151 can be a PEG10 gene having a nucleic acid sequence of SEQ ID NO: 1, the envelope protein gene 152 can be a vesicular stomatitis virus glycoprotein (“VSV-G” hereafter, wherein the “vesicular stomatitis virus” is also known as “Vesiculovirus indiana”) gene having a nucleic acid sequence of SEQ ID NO: 2, the engineered envelope protein gene 153 can be an engineered vesicular stomatitis virus glycoprotein (“eVSV-G” hereafter) gene having a nucleic acid sequence of SEQ ID NO: 3, and the target gene 154 can include an enhanced green fluorescent protein (“EGFP” hereafter) gene, an interleukin-12 (“IL-12” hereafter) or an OX40L gene. Further, the EGFP gene has a nucleic acid sequence of SEQ ID NO: 4, the IL-12 gene has a nucleic acid sequence of SEQ ID NO: 5, and the OX40L gene has a nucleic acid sequence of SEQ ID NO: 6. Furthermore, the single chain variable fragment gene 173 in the engineered envelope protein gene 153 can be an epidermal growth factor receptor (“EGFR” hereafter) single chain variable fragment gene having a nucleic acid sequence of SEQ ID NO: 7, but the present disclosure is not limited thereto.

In Step 120, a plasmid transposing step is performed, wherein the first donor plasmid 111 is transposed to a first shuttle vector, the second donor plasmid 112 is transposed to a second shuttle vector, and the third donor plasmid 113 is transposed to a third shuttle vector, wherein the first shuttle vector is transfected to a first virus-amplifying cell, the second shuttle vector is transfected to a second virus-amplifying cell, and the third shuttle vector is transfected to a third virus-amplifying cell. In detail, in the first of Step 120, the first donor plasmid 111 is transformed into a first competent cell, the second donor plasmid 112 is transformed into a second competent cell, and the third donor plasmid 113 is transformed into a third competent cell, wherein a helper plasmid of the first competent cell, a helper plasmid of the second competent cell and a helper plasmid of the third competent cell respectively express a transposase. Next, the nucleocapsid protein gene 151 and the envelope protein gene 152 located at the Tn7 site in the first donor plasmid 111 are transposed to the first shuttle vector of the first competent cell by the transposase in the first competent cell, the engineered envelope protein gene 153 located at the Tn7 site in the second donor plasmid 112 is transposed to the second shuttle vector of the second competent cell by the transposase in the second competent cell, and the target gene 154 located at the Tn7 site in the third donor plasmid 113 is transposed to the third shuttle vector of the third competent cell by the transposase in the third competent cell. After that, the first shuttle vector is obtained from the first competent cell, the second shuttle vector is obtained from the second competent cell, and the third shuttle vector is obtained from the third competent cell. Finally, the first shuttle vector is transfected to the first virus-amplifying cell, the second shuttle vector is transfected to the second virus-amplifying cell, and the third shuttle vector is transfected to the third virus-amplifying cell. Further, the first competent cell, the second competent cell and the third competent cell can be DH10Bac cells, and the first virus-amplifying cell, the second virus-amplifying cell and the third virus-amplifying cell can be Sf9 cells, but the present disclosure is not limited thereto.

In Step 130, a recombinant virus preparing step is performed, wherein the first virus-amplifying cell, the second virus-amplifying cell and the third virus-amplifying cell are cultured so as to obtain a first recombinant baculovirus, a second recombinant baculovirus and a third recombinant baculovirus. In detail, the first recombinant baculovirus is obtained from the first virus-amplifying cell, the second recombinant baculovirus is obtained from the second virus-amplifying cell, and the third recombinant baculovirus is obtained from the third virus-amplifying cell. Therefore, the first recombinant baculovirus carries the nucleocapsid protein gene 151 and the envelope protein gene 152, the second recombinant baculovirus carries the engineered envelope protein gene 153, and the third recombinant baculovirus carries the target gene 154. Further, the first recombinant baculovirus, the second recombinant baculovirus and the third recombinant baculovirus can respectively be a recombinant Autographa californica multiple nucleopolyhedrovirus (AcMNPV), but the present disclosure is not limited thereto.

In Step 140, a transducing step is performed, wherein the first recombinant baculovirus, the second recombinant baculovirus and the third recombinant baculovirus are used to infect a producer cell so as to express a nucleocapsid protein, an envelope protein, an engineered envelope protein and a target RNA, and the nucleocapsid protein, the envelope protein, the engineered envelope protein and the target RNA are self-assembled so as to form a protein-based nanoparticle, and the protein-based nanoparticle is for self-packaging and delivering mRNA. In detail, the producer cell can be a human embryonic kidney cell 293T (“HEK 293T cell” hereafter), and the target RNA can be an mRNA, but the present disclosure is not limited thereto. Further, a multiplicity of infection (MOI) of the first recombinant baculovirus to infect the producer cell can be 62 to 72, a multiplicity of infection of the second recombinant baculovirus to infect the producer cell can be 28 to 38, and a multiplicity of infection of the third recombinant baculovirus to infect the producer cell can be 45 to 55.

### Protein-Based Nanoparticle of the Present Disclosure

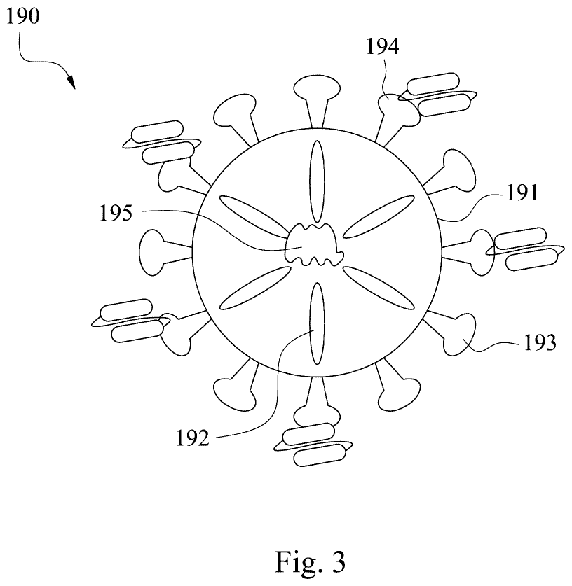

Reference is made to FIG. 3, which is a schematic view of a protein-based nanoparticle 190 of the present disclosure. The protein-based nanoparticle 190 is prepared by the method 100 for preparing protein-based nanoparticle for self-packaging and delivering mRNA of the present disclosure, and the details of the method 100 for preparing protein-based nanoparticle for self-packaging and delivering mRNA are described in the aforementioned paragraph, so that the details thereof will not be described herein again.

As shown in FIG. 3, the protein-based nanoparticle 190 is for self-packaging and delivering mRNA and includes a lipid carrier 191, a nucleocapsid protein 192, an envelope protein 193, an engineered envelope protein 194 and a target RNA 195, the target RNA 195 and the nucleocapsid protein 192 are located in the lipid carrier 191, the target RNA 195 is covered by the nucleocapsid protein 192, and the envelope protein 193 and the engineered envelope protein 194 are separately located on a surface of the lipid carrier 191. In detail, the nucleocapsid protein 192 can be a PEG10 protein having an amino acid sequence of SEQ ID NO: 8, the envelope protein 193 can be a VSV-G protein having an amino acid sequence of SEQ ID NO: 9, the engineered envelope protein 194 can be an eVSV-G protein having an amino acid sequence of SEQ ID NO: 10, and the target RNA 195 can include an EGFP mRNA, an IL-12 mRNA or an OX40L mRNA. Further, the engineered envelope protein 194 includes a single-chain variable fragment (scFv), wherein the single-chain variable fragment is a protein translated from the single chain variable fragment gene 173 shown in FIG. 2, the single-chain variable fragment can be an EGFR single-chain variable fragment having an amino acid sequence of SEQ ID NO: 11, and the single-chain variable fragment is located at N-terminus of the engineered envelope protein 194. Therefore, the protein-based nanoparticle 190 prepared by the method 100 for preparing protein-based nanoparticle for self-packaging and delivering mRNA of the present disclosure can have the cell-targeting property and the excellent transfection efficiency, and it is favorable for enhancing the treatment effect of the protein-based nanoparticle 190.

Further, the protein-based nanoparticle 190 of the present disclosure can remain stable for at least 7 months at 4° C., so that the storage costs and the shipping costs of the protein-based nanoparticle 190 of the present disclosure can be reduced, and the different usage requirements can be satisfied.

### Pharmaceutical Composition of the Present Disclosure

The pharmaceutical composition of the present disclosure includes the protein-based nanoparticle of the present disclosure, and the pharmaceutical composition is for treating a cancer. In specific, the pharmaceutical composition can be for treating a colon cancer, a brain cancer, a liver cancer or a breast cancer, and the pharmaceutical composition can be for promoting an immune response and suppressing a growth of a tumor.

Further, an effective dose of the target RNA in the protein-based nanoparticle of the present disclosure in the pharmaceutical composition of the present disclosure is 1×10⁷ copies, wherein a dosage form of the pharmaceutical composition can be a solution, an emulsion, a syrup, a powder, a tablet, a pillar, a capsule, an aerosol or an injection. Furthermore, when the dosage form of the pharmaceutical composition is the injection, the pharmaceutical composition can be administered by an intratumoral injection, a subcutaneous injection or an intravenous injection.

### EXAMPLE

The present disclosure will be further exemplified by the following specific examples so as to describe the excellent effects of the method for preparing protein-based nanoparticle for self-packaging and delivering mRNA of the present disclosure used to enhance the production efficiency of the protein-based nanoparticle, the protein-based nanoparticle of the present disclosure used to enhance the target RNA delivery efficiency and the cell-targeting property, and the treatment effect of the pharmaceutical composition of the present disclosure used to treat a cancer.

### I. Analysis of the Effect of the Method for Preparing Protein-Based Nanoparticle for Self-Packaging and Delivering mRNA Used to Enhance the Production Efficiency of the Protein-Based Nanoparticle

In order to analyze the effect of the method for preparing protein-based nanoparticle for self-packaging and delivering mRNA used to enhance the production efficiency of the protein-based nanoparticle, functional titers of the protein-based nanoparticle prepared by the method for preparing protein-based nanoparticle for self-packaging and delivering mRNA of the present disclosure and the nanoparticle prepared by the current method of plasmid transfection used to transfect the HEK 293T cells are analyzed. In the present experiment, Example 1 and Comparative example 1 are used, wherein the protein-based nanoparticle prepared by the method for preparing protein-based nanoparticle for self-packaging and delivering mRNA of the present disclosure is analyzed in Example 1, and the nanoparticle prepared by the current method of plasmid transfection is analyzed in Comparative example 1.

In specific, Example 1 and Comparative example 1 are performed by using the first donor plasmid 111 and the third donor plasmid 113 shown in FIG. 2, wherein the nucleocapsid protein gene 151 of the first donor plasmid 111 is the PEG10 gene, the envelope protein gene 152 of the first donor plasmid 111 is the VSV-G gene, and the target gene 154 of the third donor plasmid 113 is the EGFP gene. Further, the first recombinant baculovirus and the third recombinant baculovirus of Example 1 are prepared by the method 100 for preparing protein-based nanoparticle for self-packaging and delivering mRNA of the present disclosure, so that the details of the method 100 for preparing protein-based nanoparticle for self-packaging and delivering mRNA will not be described herein again.

Further, in Example 1, the first recombinant baculovirus and the third recombinant baculovirus are cotransduced to 2×10⁷ HEK 293T cells, wherein the multiplicity of infection of the first recombinant baculovirus is 50, and the multiplicity of infection of the third recombinant baculovirus is 50. In Comparative example 1, the first donor plasmid 111 (50 μg) and the third donor plasmid 113 (50 μg) are cotransfected to 2×10⁷ HEK 293T cells by the Lipofectamine 3000 transfection reagent. Next, the HEK 293T cells of Example 1 and the HEK 293T cells of Comparative example 1 are respectively cultured at 37° C. for 48 hours, a cell culture supernatant of the HEK 293T cells of Example 1 and a cell culture supernatant of the HEK 293T cells of Comparative example 1 are respectively collected, and the protein-based nanoparticle in the cell culture supernatant of Example 1 and the nanoparticle in the cell culture supernatant of Comparative example 1 are purified. Next, the protein-based nanoparticle of Example 1 is diluted 20 times, 40 times, and 200 times and then transfected to the HEK 293T cells, and the nanoparticle of Comparative example 1 is diluted 20 times, 40 times, and 200 times and then transfected to other HEK 293T cells. After that, the HEK 293T cells of Example 1 and the HEK 293T cells of Comparative example 1 are respectively cultured for 24 hours, and the EGFP expressions of the HEK 293T cells of Example 1 and Comparative example 1 are quantified by a flow cytometry. Finally, the functional titers of the protein-based nanoparticle of Example 1 and the nanoparticle of Comparative example 1 are calculated by a functional titer formula. The functional titer formula is shown as follows:

Functional titer (TU/mL)=[proportion of cells expressing fluorescent signals (%)×number of cells used for transfection (cell)×dilution factor of protein-based nanoparticle×1000]/volume of protein-based nanoparticle added to transfect the cells (mL).

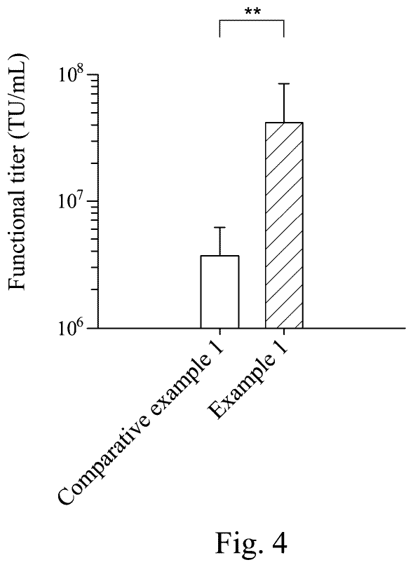

Reference is made to FIG. 4, which shows the results of the functional titer of the nanoparticle of Comparative example 1 and the protein-based nanoparticle of Example 1. Specifically, in FIG. 4, the mark “**” represents a statistical difference in comparison with Comparison example 1 (p<0.01).

As shown in FIG. 4, the protein-based nanoparticle of Example 1 is 11.3 times the functional titer of the nanoparticle of Comparison example 1. The result shows that the production efficiency of the protein-based nanoparticle can be effectively enhanced by the method for preparing protein-based nanoparticle for self-packaging and delivering mRNA of the present disclosure. Further, a large number of transfection reagents can be omitted to use in the method for preparing protein-based nanoparticle for self-packaging and delivering mRNA of the present disclosure, the protein-based nanoparticle of the present disclosure can be effectively enhanced to 11 times compared with the production efficiency of the nanoparticle prepared by the current method of plasmid transfection, and it is favorable for reducing the production costs of the protein-based nanoparticle.

### II. Analysis of the Composition of the Protein-Based Nanoparticle of the Present Disclosure

In order to analyze the composition of the protein-based nanoparticle of the present disclosure, the protein expressions of PEG10, VSV-G and GP64 in the protein-based nanoparticle of Example 1 are analyzed by Western blot analysis in the present experiment. In detail, GP64 is a protein required for baculovirus to infect a cell or spread between the cells. Hence, if the protein expression of GP64 is not detected in the protein-based nanoparticle of Example 1, it indicates that the protein-based nanoparticle of Example 1 is not infectious, and it is favorable for the application in clinical medicine and other fields.

In the present experiment, a cell lysate of the producer cells in Example 1 (that is, the HEK 293T cells transduced with the recombinant baculoviruses, which is called “Transduced group” hereafter) and a cell lysate of the HEK 293T cells that are not transduced with any recombinant baculoviruses (“Untransduced group” hereafter) are simultaneously analyzed by Western blot analysis so as to analyze the protein expressions of PEG10, VSV-G and GP64 in the cell lysates, and Transduced group and Untransduced group are used to compare with the protein composition of the protein-based nanoparticle of Example 1.

Further, the operation details of Western blot analysis are well-known in the art and can be adjusted according to the experimental requirements, so the detailed steps thereof will not be described herein.

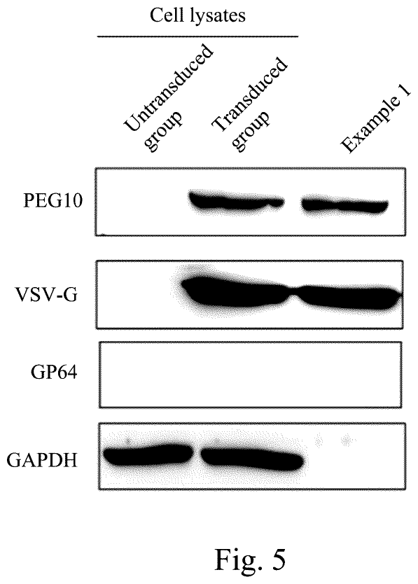

Reference is made to FIG. 5, which shows the results of Western blot analysis of the cell lysate of Untransduced group, the cell lysate of Transduced group and the protein-based nanoparticle of Example 1. As shown in FIG. 5, compared to the cell lysate of Untransduced group, a larger number of the PEG10 proteins and the VSV-G proteins are detected in the cell lysate of Transfected group and the protein-based nanoparticle of Example 1. The result shows that the producer cells can express a larger number of the nucleocapsid proteins and the envelope proteins after the producer cells are transduced with the recombinant baculoviruses, and it is favorable for forming the protein-based nanoparticle. Further, the GP64 proteins are not detected in the cell lysate of Untransduced group, the cell lysate of Transduced group and the protein-based nanoparticle of Example 1. The result shows that the producer cells transduced by the recombinant baculoviruses in the method for preparing protein-based nanoparticle for self-packaging and delivering mRNA of the present disclosure are not infectious, and the protein-based nanoparticle prepared by the method for preparing protein-based nanoparticle for self-packaging and delivering mRNA of the present disclosure are not infectious, so that the protein-based nanoparticle can have excellent safety in use. Therefore, the method for preparing protein-based nanoparticle for self-packaging and delivering mRNA of the present disclosure can have excellent application values in fields such as drug development.

### III. Analysis of the Effect of Different Proportions of Engineered Envelope Proteins Used to Enhance the Target RNA Delivery Efficiency of the Protein-Based Nanoparticle of the Present Disclosure

In order to analyze the effect of different proportions of engineered envelope proteins used to enhance the target RNA delivery efficiency of the protein-based nanoparticle of the present disclosure, the efficiencies of the protein-based nanoparticles of Example 2 to Example 4 and the nanoparticle of Comparative example 2 for transfecting the murine CT26 colorectal carcinoma cells (“CT26 cells” hereafter) are analyzed in the present experiment, wherein the surfaces of the lipid carriers of the protein-based nanoparticles of Example 2 to Example 4 have different proportions of the engineered envelope proteins, and the surface of the lipid carrier of the nanoparticle of Comparative example 2 does not have the engineered envelope protein, so that the optimal proportion of the engineered envelope protein distributed on the surface of the lipid carrier can be analyzed.

In specific, the nucleocapsid protein gene 151 and the envelope protein gene 152 shown in FIG. 2 are respectively integrated into two pFastBac-dual plasmids to form two first donor plasmids respectively, and the two first donor plasmids respectively include the nucleocapsid protein gene 151 or the envelope protein gene 152, and then the first recombinant baculovirus including the nucleocapsid protein gene 151 (“Recombinant baculovirus 1-1” hereafter), the first recombinant baculovirus including the envelope protein gene 152 (“Recombinant baculovirus 1-2” hereafter), the second recombinant baculovirus including the engineered envelope protein gene 153 (“Recombinant baculovirus 2” hereafter) and the third recombinant baculovirus including the target gene 154 (“Recombinant baculovirus 3” hereafter) are prepared by the two first donor plasmids, the second donor plasmid 112 and the third donor plasmid 113 according to the method 100 for preparing protein-based nanoparticle for self-packaging and delivering mRNA of FIG. 1. Next, Recombinant baculovirus 1-1, Recombinant baculovirus 1-2, Recombinant baculovirus 2 and Recombinant baculovirus 3 are cotransduced to the HEK 293T cells at the multiplicity of infections shown in Table 1 so as to obtain the protein-based nanoparticles of Example 2 to Example 4 and the nanoparticle of Comparative example 2. Further, the details of the method 100 for preparing protein-based nanoparticle for self-packaging and delivering mRNA are described in the aforementioned paragraph, so that the details thereof will not be described herein again. Furthermore, in the present experiment, the nucleocapsid protein gene 151 is the PEG10 gene, the envelope protein gene 152 is the VSV-G gene, the engineered envelope protein gene 153 is the eVSV-G gene, and the target gene 154 is the EGFP gene.

TABLE 1

| | Multiplicity of infection | | | |
|---|---|---|---|---|
| | | | | Comparative |
| | Example 2 | Example 3 | Example 4 | example 2 |
| Recombinant | 50 | 50 | 50 | 50 |
| baculovirus 1-1 | | | | |
| Recombinant | 0 | 16.7 | 33.3 | 50 |
| baculovirus 1-2 | | | | |
| Recombinant | 50 | 33.3 | 16.7 | 0 |
| baculovirus 2 | | | | |
| Recombinant | 50 | 50 | 50 | 50 |
| baculovirus 3 | | | | |
| Proportion of eVSV-G | 100% | 67% | 33% | 0% |
| protein on the surface | | | | |
| of the lipid carrier | | | | |

As shown in Table 1, in the present experiment, the producer cells of Example 2 are not infected by Recombinant baculovirus 1-2, and the producer cells of Comparative example 2 are not infected by Recombinant baculovirus 2. Therefore, the surface of the lipid carrier of the protein-based nanoparticle of Example 2 has 100% eVSV-G protein, the surface of the lipid carrier of the protein-based nanoparticle of Example 3 has 67% eVSV-G protein and 33% VSV-G protein, the surface of the lipid carrier of the protein-based nanoparticle of Example 4 has 33% eVSV-G protein and 67% VSV-G protein, and the surface of the nanoparticle of Comparative example 2 has 0% eVSV-G protein.

After that, the protein-based nanoparticles of Example 2 to Example 4 and the nanoparticle of Comparative example 2 are respectively transfected to the CT26 cells at a dose of 10 EGFP mRNA copies/cell, and then the CT26 cells expressing EGFP fluorescence signals are measured by the flow cytometry after transfected for 48 hours so as to analyze the functional titers and the transfection efficiencies of the protein-based nanoparticles of Example 2 to Example 4 and the nanoparticle of Comparative example 2. Further, the functional titers of the protein-based nanoparticles of Example 2 to Example 4 and the nanoparticle of Comparative example 2 are calculated by the aforementioned functional titer formula, and the details thereof are shown in the foregoing description and not described again.

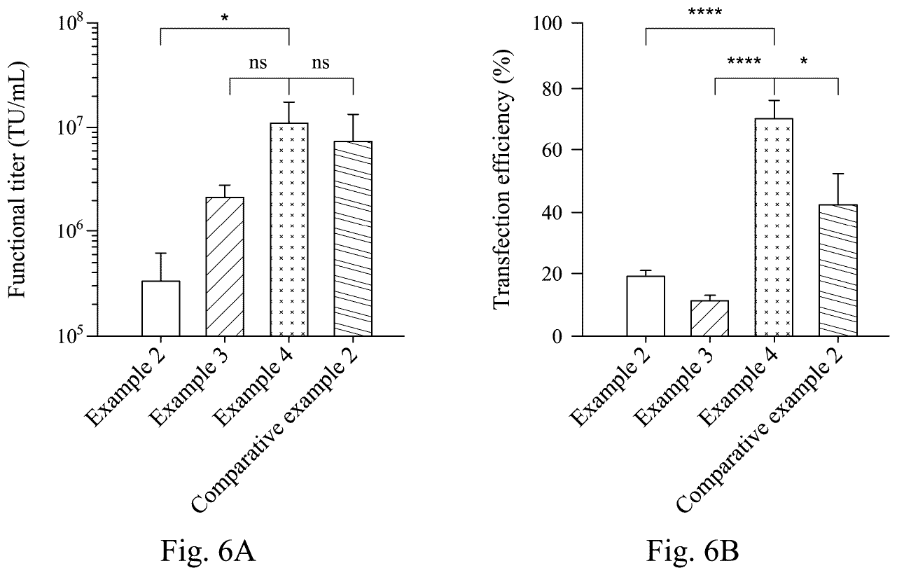

Reference is made to FIG. 6A and FIG. 6B. FIG. 6A shows the results of the functional titer of the protein-based nanoparticles of Example 2 to Example 4 and the nanoparticle of Comparative example 2. FIG. 6B shows the results of the transfection efficiency of the protein-based nanoparticles of Example 2 to Example 4 and the nanoparticle of Comparative example 2 used to transfect the CT26 cells. Specifically, in FIG. 6A and FIG. 6B, the mark “ns” represents there is no statistical difference in comparison with the protein-based nanoparticle of Example 4. The mark “*” represents a statistical difference in comparison with the protein-based nanoparticle of Example 4 (p<0.05), and the mark “****” represents p<0.0001. Further, in FIG. 6B, “Transfection efficiency” indicated on the vertical axis represents the proportion of the CT26 cells expressing the EGFP fluorescence signals.

As shown in FIG. 6A, all of the protein-based nanoparticles of Example 2 to Example 4 have great functional titers, and the functional titer of the protein-based nanoparticle of Example 4 is better than the functional titer of the nanoparticle of Comparative example 2. The result shows that the protein-based nanoparticle can have great transfection effect when the surface of the protein-based nanoparticle has the engineered envelope protein. As shown in FIG. 6B, the protein-based nanoparticles of Example 2 to Example 4 can transfect to the CT26 cells, wherein the transfection efficiency of the protein-based nanoparticle of Example 4 is significantly enhanced compared with the transfection efficiency of the nanoparticle of Comparative example 2. The result shows that the protein-based nanoparticle can have excellent transfection efficiency when the proportion of eVSV-G protein on the surface of the lipid carrier is approximately 33%. Therefore, the protein-based nanoparticle of the present disclosure can have excellent target RNA delivery efficiency.

### IV. Analysis of the Efficiencies of the Protein-Based Nanoparticles of the Present Disclosure Used to Transfect Different Types of Cells

In the present experiment, the nanoparticles of Comparative example 2 and the protein-based nanoparticles of Example 4 are respectively transfected to the HEK 293T cell, the CT26 cells, the U87 human glioblastoma astrocytoma cells, the MC38 murine colon adenocarcinoma cells (“MC38 cells” hereafter), the Hepa1-6 murine hepatoma cells, the Huh7 human liver carcinoma cells, the Hep3B human hepatoma cells and the 4T1 mouse mammary carcinoma cells, and then the proportions of the cells expressing EGFP fluorescence signals are measured by the flow cytometry after transfected for 48 hours, so that the efficiencies of the protein-based nanoparticles of the present disclosure used to transfect different types of cells can be analyzed. Further, both of the transfection doses of the nanoparticles of Comparative example 2 and the protein-based nanoparticles of Example 4 are 10 EGFP mRNA copies/cell.

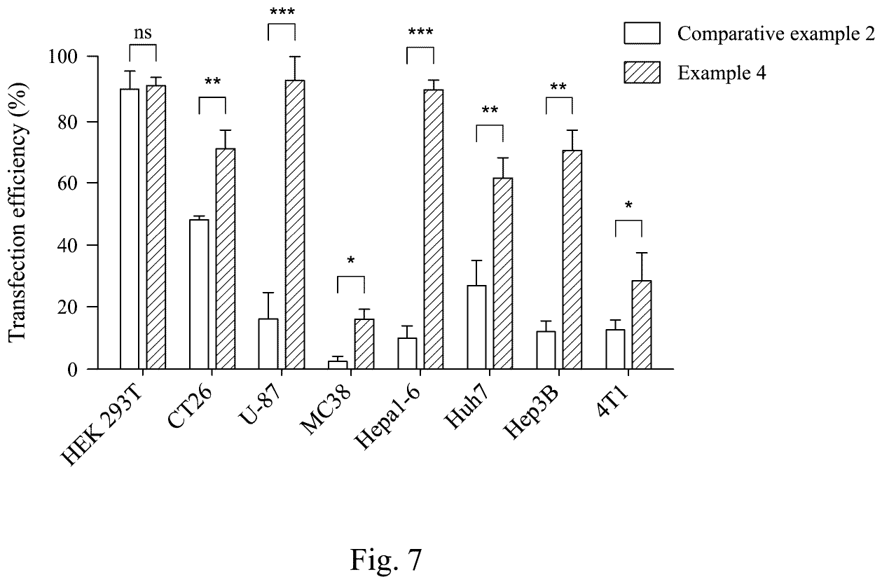

Reference is made to FIG. 7, which shows the results of the transfection efficiency of the protein-based nanoparticles of Example 2 to Example 4 and the nanoparticles of Comparative example 2 used to transfect different types of cells. Specifically, in FIG. 7, the mark “ns” represents there is no statistical difference in comparison with Comparative example 2. The mark “*” represents a statistical difference in comparison with Comparative example 2 (p<0.05), the mark “**” represents p<0.01, and the mark “***” represents p <0.001.

As shown in FIG. 7, compared with the nanoparticles of Comparative Example 2, the protein-based nanoparticles of Example 4 have excellent transfection efficiencies when they are transfected to different types of cells. The result shows that the protein-based nanoparticle of the present disclosure has excellent target RNA delivery efficiency and the potential to treat different types of cancers. Further, compared with other types of cells, the MC38 cells are less sensitive to the transfection of the protein-based nanoparticles of Example 4 because the MC38 cells express a low level of EGFR, wherein each of the protein-based nanoparticles of Example 4 has the EGFR single-chain variable fragment on the surface of the lipid carrier. According to the above, the protein-based nanoparticle of the present disclosure has the cell-targeting property and the excellent target RNA delivery efficiency.

### V. Analysis of the Effect of the Protein-Based Nanoparticle of the Present Disclosure Used to Induce a Cell to Express a Target Protein

In order to analyze the effect of the protein-based nanoparticle of the present disclosure used to induce the cell to express the target protein, the effects of different doses of the protein-based nanoparticles of Example 5 used to induce the CT26 cells to secrete the target proteins after being transfected to the CT26 cells are analyzed by the enzyme-linked immunosorbent assay (ELISA) so as to evaluate the optimal transfection dose of the protein-based nanoparticle of Example 5 and the effect thereof to increase the expressions of the target proteins. In the present experiment, the protein-based nanoparticle of Example 5 is prepared by the first donor plasmid 111, the second donor plasmid 112 and the third donor plasmid 113 shown in FIG. 2 according to the method 100 for preparing protein-based nanoparticle for self-packaging and delivering mRNA of FIG. 1, and the nanoparticle of Comparative example 3 is prepared by the first donor plasmid 111 and the third donor plasmid 113 according to the method 100 for preparing protein-based nanoparticle for self-packaging and delivering mRNA of FIG. 1, so that the effect of engineered envelope protein in enhancing the immune responses induced by the protein-based nanoparticle of the present disclosure can be further analyzed. Further, the details of the protein-based nanoparticle of Example 5 and the nanoparticle of Comparative example 3 are described in the aforementioned paragraph, so that the details thereof will not be described herein again.

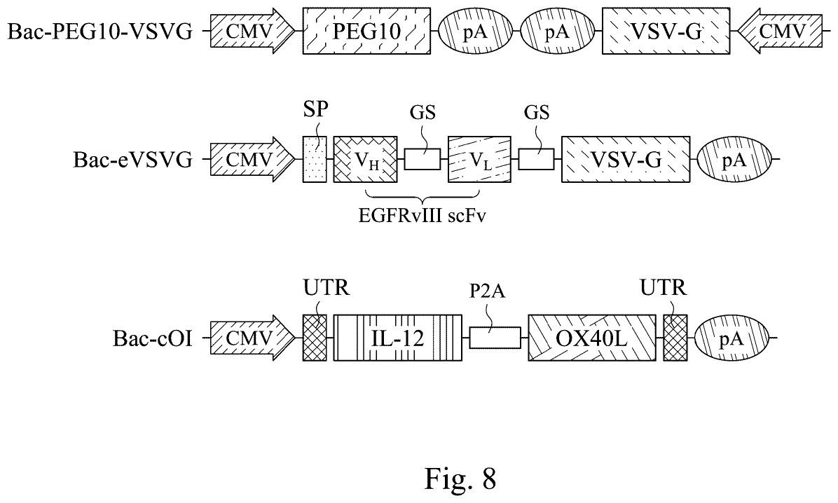

Reference is made to FIG. 8, which is a schematic view of genes carried by the first recombinant baculovirus (Bac-PEG10-VSVG), the second recombinant baculovirus (Bac-eVSVG) and the third recombinant baculovirus (Bac-cOI) in Example 5.

In Example 5, the nucleocapsid protein gene is the PEG10 gene, the envelope protein gene is the VSV-G gene, the engineered envelope protein gene includes the EGFR single-chain variable fragment gene (EGFRvIII scFv) and the VSV-G gene, and the target gene includes the IL-12 gene and the OX40L gene. Further, the IL-12 gene and the OX40L gene are connected by a P2A linker gene, and the target gene has a nucleic acid sequence of SEQ ID NO: 12. Furthermore, in Example 5, the first promoter, the second promoter, the third promoter and the fourth promoter are CMV promoters, and both the two linker genes in the engineered envelope protein gene are GS linker genes. Moreover, in FIG. 8, “pA” represents the poly-A tail, “SP” represents the signal peptide gene, “VH” represents the heavy chain variable region, “VL” represents the light chain variable region, and “UTR” represents the untranslated region.

Further, in Example 5, the multiplicity of infection of the first recombinant baculovirus is 66.7, the multiplicity of infection of the second recombinant baculovirus is 33.3, and the multiplicity of infection of the third recombinant baculovirus is 50. Furthermore, in Comparative example 3, the multiplicity of infection of the first recombinant baculovirus is 50, and the multiplicity of infection of the third recombinant baculovirus is 50.

Next, the protein-based nanoparticles of Example 5 are transfected to the CT26 cells at target RNA doses of 0 copies/cell, 5 copies/cell, 10 copies/cell, 15 copies/cell and 20 copies/cell, and then the concentrations of IL-12 of the CT26 cells transfected with different target RNA doses are analyzed by the enzyme-linked immunosorbent assay after the transfections of the protein-based nanoparticles of Example 5 for 24 hours, 48 hours, 72 hours and 96 hours, so that the optimal transfection dose of the protein-based nanoparticle of Example 5 can be analyzed.

Further, the operation details of the enzyme-linked immunosorbent assay are well-known in the art and can be adjusted according to the experimental requirements, so the detailed steps thereof will not be described herein again.

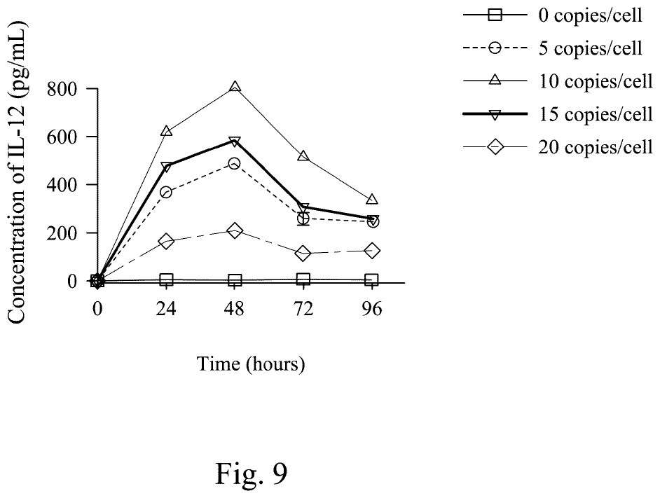

Reference is made to FIG. 9, which shows the results of concentration of IL-12 at different time points after the CT26 cells are transfected with different doses of the protein-based nanoparticles of Example 5. As shown in FIG. 9, when a transfection dose of the protein-based nanoparticles of Example 5 is between 5 copies/cell and 20 copies/cell, the CT26 cells can be effectively induced to secrete IL-12 by the protein-based nanoparticles of Example 5. Further, when the transfection dose of the protein-based nanoparticles of Example 5 is between 5 copies/cell and 15 copies/cell, the protein-based nanoparticles of Example 5 can have an excellent effect in inducing the CT26 cells to secrete IL-12. According to the above, when the protein-based nanoparticle of the present disclosure is transfected to the cells at the transfection dose of 5 copies/cell and 15 copies/cell, the protein-based nanoparticle of the present disclosure can have an excellent effect in inducing the cells to express the target proteins.

### VI. Analysis of the Effect of Cells Transfected With the Protein-Based Nanoparticle of the Present Disclosure Used to Stimulate Neighboring Immune Cells to Produce Immune Responses

In the present experiment, the nanoparticle of Comparative example 3and the protein-based nanoparticle of Example 5 are respectively transfected to the CT26 cells at the dose of 10 copies/cell, and then the CT26 cells are cocultured with the mouse splenocytes, so that the effect of the cells transfected with the protein-based nanoparticle of the present disclosure used to stimulate the neighboring immune cells to produce the immune responses can be analyzed.

In specific, the CT26 cells and the mouse splenocytes are cocultured by the Transwell method, wherein the CT26 cells transfected with the nanoparticle of Comparative example 3 or the protein-based nanoparticle of Example 5 are cultured in the cell culture dish, and the mouse splenocytes are cultured in the Transwell chamber, so that cytokines secreted by the CT26 cells can pass through the porous membrane under the Transwell chamber and then enter from the cell culture dish into the Transwell chamber so as to induce the mouse splenocytes to produce the immune responses.

### 1. Analysis of the Effect of the Cells Transfected With the Protein-Based Nanoparticle of the Present Disclosure Used to Stimulate the Neighboring Immune Cells to Express the Cytokines

After the nanoparticle of Comparative example 3 or the protein-based nanoparticle of Example 5 are transfected to the CT26 cells for 24 hours, 48 hours, 72 hours and 96 hours, the cell culture supernatants at different time points are collected, and the concentration changes of IL-12 and OX40L in the cell culture supernatants are analyzed by the enzyme-linked immunosorbent assay, so that the effect of the protein-based nanoparticle of the present disclosure used to induce the CT26 cells to express IL-12 and OX40L can be analyzed. Further, in the present experiment, the CT26 cells untransfected with the protein-based nanoparticle are served as Control group 1 so as to further analyze the effect of the CT26 cells transfected with the nanoparticle of Comparative example 3 or the protein-based nanoparticle of Example 5 used to express the target proteins.

In detail, the CT26 cells will be induced by the IL-12 mRNA and the OX40L mRNA of the protein-based nanoparticle of Example 5 to express IL-12 and OX40L after the CT26 cells are transfected with the protein-based nanoparticle of Example 5, wherein IL-12 is a proinflammatory cytokine used to promote the proliferation of Type 1 T helper (Th1) cells and the secretion of the IFN-γ, the OX40L is a ligand for OX40 and is stably expressed on the antigen-presenting cells (such as macrophages and activated B lymphocytes), and the interaction between the OX40L and the OX40 will activate CD4⁺ T cells and promote the development of memory T cells. Therefore, by analyzing the concentrations of IL-12 and the concentrations of OX40L at different time points, the timeliness of the target protein expressions induced by the target RNA in the cells transfected with the protein-based nanoparticle of the present disclosure can be evaluated.

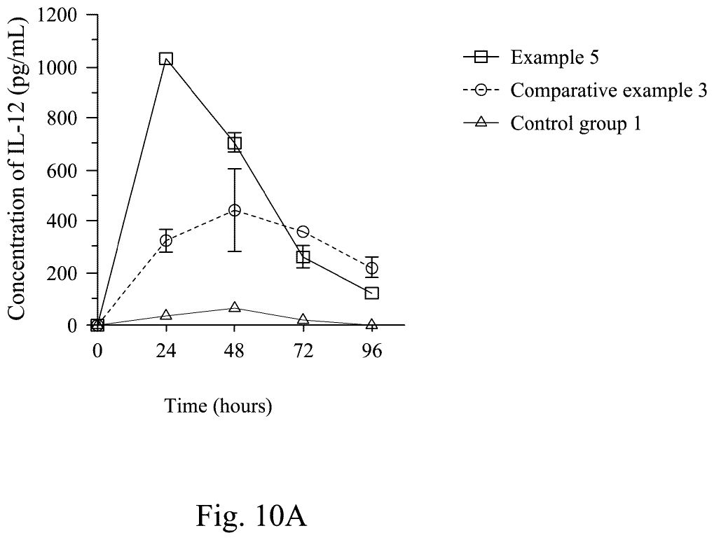

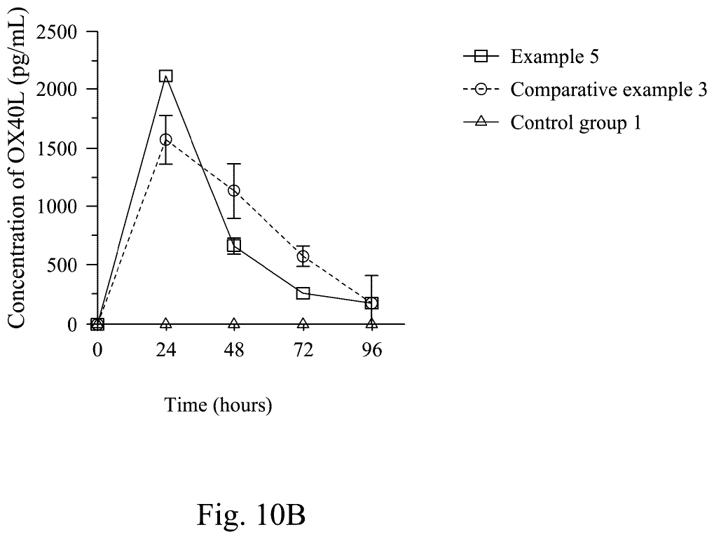

Reference is made to FIG. 10A and FIG. 10B. FIG. 10A shows the results of concentration of IL-12 at different time points after the CT26 cells are respectively transfected with the nanoparticle of Comparative example 3 and the protein-based nanoparticle of Example 5. FIG. 10B shows the results of concentration of OX40L at different time points after the CT26 cells are respectively transfected with the nanoparticle of Comparative example 3 and the protein-based nanoparticle of Example 5. As shown in FIG. 10A, after the CT26 cells are transfected with the protein-based nanoparticle of Example 5 or the nanoparticle of Comparative example 3 for 24 hours and 48 hours, the concentrations of IL-12 of Example 5 are greater than 600 pg/mL and are much more than the concentrations of IL-12 of Control group 1 or the concentrations of IL-12 of Comparative example 3. As further shown in FIG. 10B, after the CT26 cells are transfected with the protein-based nanoparticle of Example 5 or the nanoparticle of Comparative example 3 for 24 hours, the concentration of OX40L of Example 5 is greater than the concentration of OX40L of Control group 1 or the concentration of OX40L of Comparative example 3. According to the above, the cells transfected with the protein-based nanoparticle of the present disclosure can be effectively induced to express the target proteins in high levels within 48 hours.

Further, in order to analyze the immunostimulatory effect of IL-12 and OX40L on the neighboring immune cells, Dynabeads are added in the Transwell chamber and cocultured with the mouse splenocytes after the CT26 cells transfected with the nanoparticle of Comparative example 3 or the protein-based nanoparticle of Example 5 and the mouse splenocytes are cocultured for 24 hours, and concentrations of interferon-γ (“IFN-γ” hereafter) and concentrations of tumor necrosis factor-α (“TNF-α” hereafter) in the cell culture supernatants are analyzed by the enzyme-linked immunosorbent assay after the Dynabeads, the CT26 cells and the mouse splenocytes are cocultured for 24 hours, 48 hours and 72 hours. Furthermore, in the present experiment, the CT26 cells that are untransfected with the protein-based nanoparticle and the mouse splenocytes that are not cultured with the Dynabeads are served as Control group 1, and the CT26 cells that are untransfected with the protein-based nanoparticle and the mouse splenocytes that are cocultured with the Dynabeads are served as Comparison group 1, so that the effect of the target protein secreted by the CT26 cells transfected with the nanoparticle of Comparative example 3 or the protein-based nanoparticle of Example 5 used to stimulate the neighboring immune cells to express the cytokines can be further analyzed.

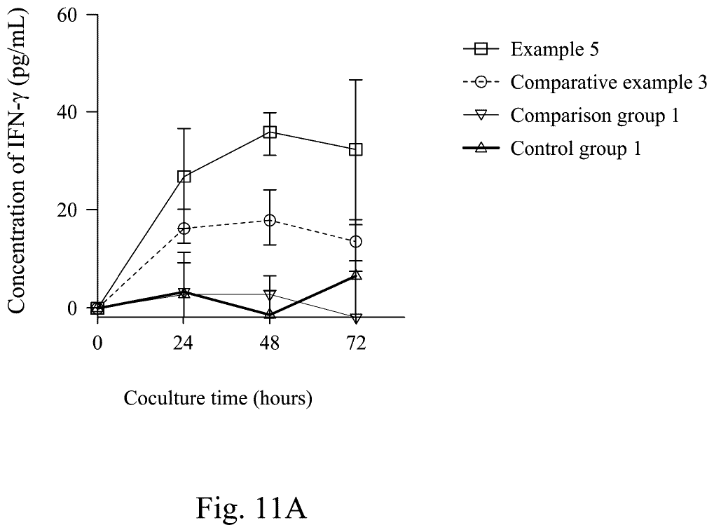

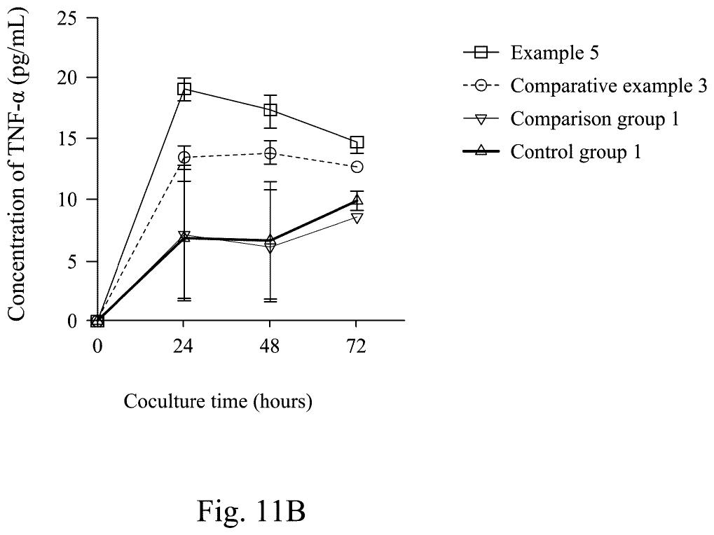

Reference is made to FIG. 11A and FIG. 11B. FIG. 11A shows the results of the concentration of IFN-γ at different time points after the CT26 cells are respectively transfected with the nanoparticle of Comparative example 3 and the protein-based nanoparticle of Example 5. FIG. 11B shows the results of concentration of TNF-α at different time points after the CT26 cells are respectively transfected with the nanoparticle of Comparative example 3 and the protein-based nanoparticle of Example 5. As shown in FIG. 11A and FIG. 11B, compared with the mouse splenocytes of Control group 1 and the mouse splenocytes of Comparison group 1, the mouse splenocytes of Comparative example 3 and the mouse splenocytes of Example 5 are affected by IL-12 and OX40L secreted by the CT26 cells to secrete a large number of IFN-γ and TNF-α, wherein the concentrations of IFN-γ and the concentrations of TNF-α of Example 5 within 72 hours are relatively higher than those of Control group 1,Comparison group 1 and Comparative example 3. According to the above, after the cells are transfected with the protein-based nanoparticle of the present disclosure, the neighboring immune cells can be effectively induced to express cytokines within 72 hours.

### 2. Analysis of the Immunostimulatory Effect of Cells Transfected With the Protein-Based Nanoparticle of the Present Disclosure on Neighboring Immune Cells

In the present experiment, the CT26 cells transfected with the protein-based nanoparticle of Example 5, the mouse splenocytes and the Dynabeads are cocultured for 6 days, and then the proportion of the CD4⁺ T cells expressing CD69 and the proportion of the CD8⁺ T cell expressing CD69 in the mouse splenocytes are analyzed by the flow cytometry, so that the effect of the protein-based nanoparticle of the present disclosure used to induce the immune responses can be further analyzed. Further, the CD69 is a T-cell activation marker.

Further, in the present experiment, Control group 1 is a group of the CT26 cells untransfected with the protein-based nanoparticle of the present disclosure and the mouse splenocytes not cultured with the Dynabeads, Comparison group 1 is a group of the CT26 cells untransfected with the protein-based nanoparticle of the present disclosure and the mouse splenocytes cocultured with the Dynabeads, and Comparative example 3 is a group of the CT26 cells transfected with the nanoparticle of Comparative example 3 and the mouse splenocytes cocultured with the Dynabeads.

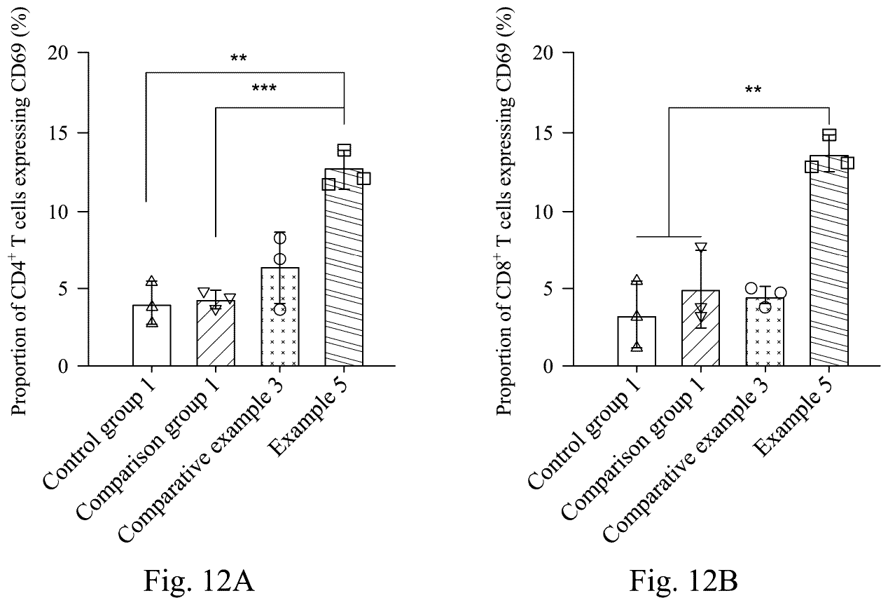

Reference is made to FIG. 12A and FIG. 12B. FIG. 12A shows the results of the proportion of CD4⁺ T cells expressing CD69 after the CT26 cells are respectively transfected with the nanoparticle of Comparative example 3 and the protein-based nanoparticle of Example 5. FIG. 12B shows the results of the proportion of CD8⁺ T cells expressing CD69 after the CT26 cells are respectively transfected with the nanoparticle of Comparative example 3 and the protein-based nanoparticle of Example 5. Specifically, in FIG. 12A and FIG. 12B, the mark “**” represents a statistical difference in comparison with Example 5 (p<0.01), and the “***” mark represents p<0.001.

As shown in FIG. 12A, compared with Control group 1, Comparison group 1 and Comparative example 3, there are more CD4⁺ T cells expressing CD69 (13% of CD4⁺ T cells expressing CD69) in Example 5. As further shown in FIG. 12B, compared with Control group 1, Comparison group 1 and Comparative example 3, there are more CD8⁺ T cells expressing CD69 (approximately 15% of CD8⁺ T cells expressing CD69) in Example 5. According to the above, the T cell can be effectively activated by the protein-based nanoparticle of the present disclosure. More particularly, the CD4⁺ T cells and the CD8⁺ T cell can be effectively activated by the protein-based nanoparticle of the present disclosure.

### VII. Analysis of the Effect of the Pharmaceutical Composition of the Present Disclosure Used to Treat a Cancer

In order to analyze the effect of the pharmaceutical composition of the present disclosure used to treat the cancer, the mouse tumor models are administrated with the pharmaceutical composition of the present disclosure in Example 6 so as to evaluate the treatment effect of the pharmaceutical composition of the present disclosure on the cancer, wherein the pharmaceutical composition of the present disclosure includes the protein-based nanoparticle of Example 5. Further, the present experiment includes Example 7, wherein the mouse tumor models are administrated with the pharmaceutical composition of the present disclosure and a chemotherapy drug so as to analyze the synergistic therapy of the pharmaceutical composition of the present disclosure and the chemotherapy drug. In the present experiment, the pharmaceutical composition of the present disclosure includes the protein-based nanoparticle of Example 5, wherein an effective dose of the target RNA in the protein-based nanoparticle of Example 5 in the pharmaceutical composition is 1×10⁷ copies, and the chemotherapy drug of Example 7 is oxaliplatin.

In the present experiment, 1×10⁵ of CT26 cells are subcutaneous injected to each of BALB/c mice so as to induce the BALB/c mice to generate tumors. The BALB/c mice are divided into four groups namely Control group 2(six mice), Comparison group 2 (six mice), Example 6 (six mice) and Example 7 (six mice) for different treatments after the tumor size of each of the BALB/c mice is 50 mm³ (Day 0), wherein the BALB/c mice in Control group 2 are administrated with 100 μL of phosphate buffered saline (PBS) by the intratumoral injection on Day 1 of the experiment, the BALB/c mice in Comparison group 2 are administrated with 3 mg/Kg of oxaliplatin by the intratumoral injection on Day 1, Day 3, Day 5 and Day 7 of the experiment, the BALB/c mice in Example 6 are administrated with the pharmaceutical composition of the present disclosure by the intratumoral injection on Day 1,Day 3, Day 5 and Day 7 of the experiment, and the BALB/c mice in Example 7 are administrated with 3 mg/Kg of oxaliplatin by the intratumoral injection on Day 1 of the experiment and administrated with the pharmaceutical composition of the present disclosure by the intratumoral injection on Day 3, Day 5, Day 7 and Day 9 of the experiment. The tumor growths of the mouse tumor models in each group are regularly measured to analyze the treatment effect of each group within 30 days of the experiment.

Further, the blood samples of the mouse tumor models are collected at 24 hours and 72 hours after each of the groups is administrated with respective treatment dosages, and the concentrations of IFN-γ and the concentrations of TNF-α in the serums of the blood samples are analyzed by the enzyme-linked immunosorbent assay, so that the changes of the immune responses of the mouse tumor models of each group after being administrated with respective treatment dosages can be evaluated. In detail, the blood sample of Control group 2 is collected and analyzed on Day 2 and Day 4 of the experiment, the blood sample of Comparison group 2 and the blood sample of Example 6 are collected and analyzed on Day 8 and Day 10 of the experiment, and the blood sample of Example 7 is collected and analyzed on Day 10 and Day 12 of the experiment.

### 1. Analysis of the Effect of the Pharmaceutical Composition of the Present Disclosure Used to Inhibit a Tumor Growth

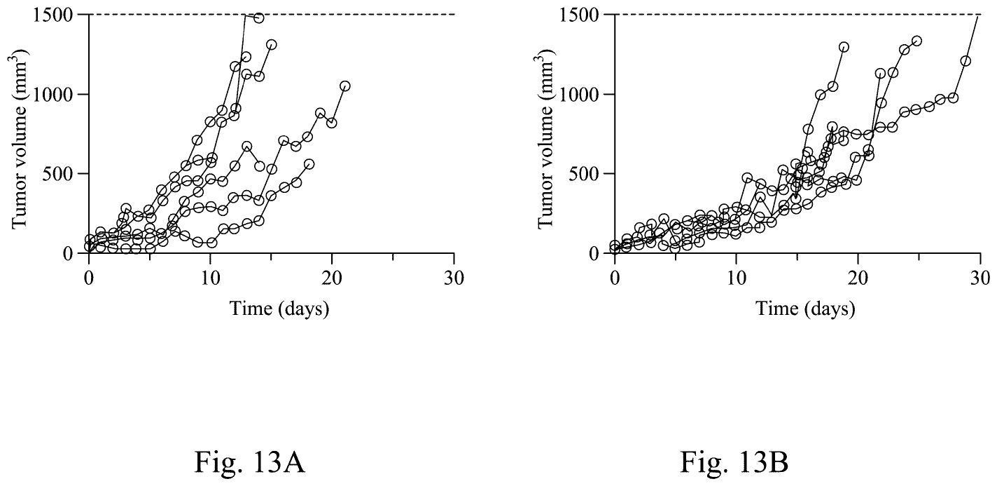

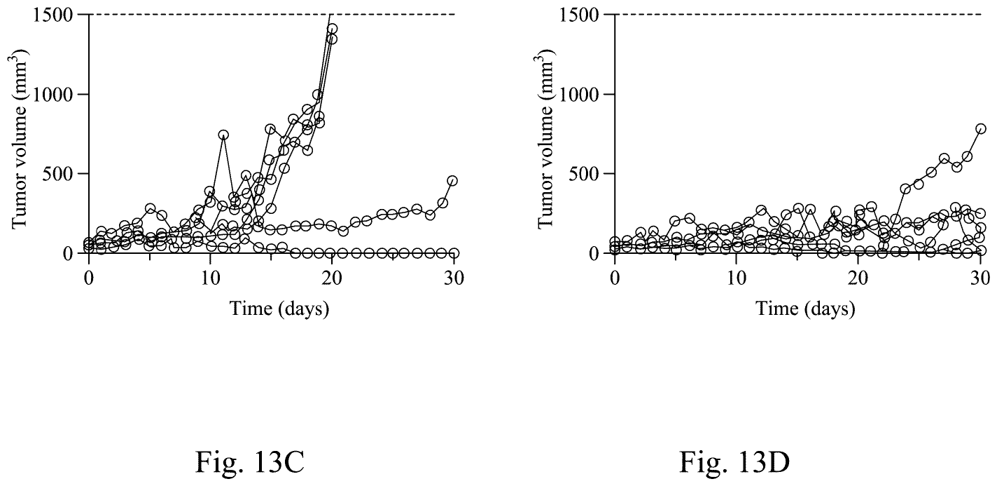

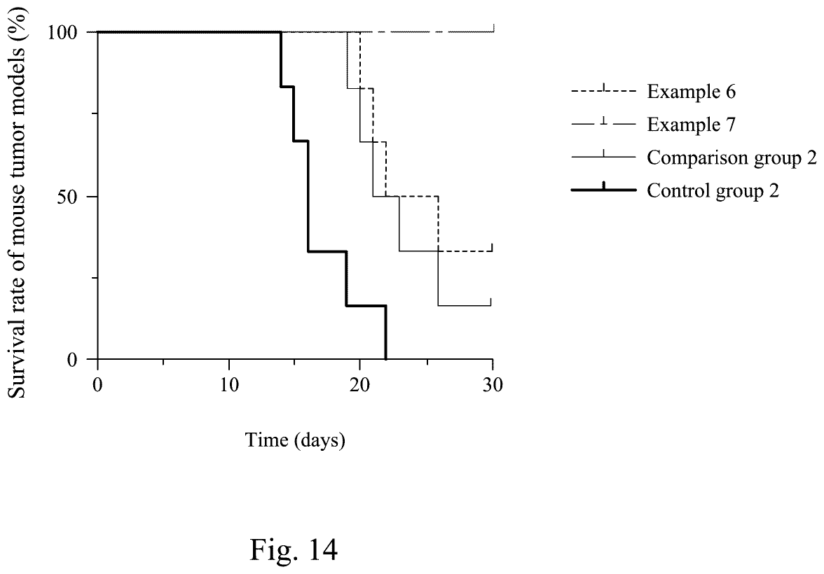

Reference is made to FIG. 13A to FIG. 14. FIG. 13A shows the tumor-growing curves of the mouse tumor models of Control group 2 within 30 days. FIG. 13B shows the tumor-growing curves of the mouse tumor models of Comparison group 2 within 30 days. FIG. 13C shows the tumor-growing curves of the mouse tumor models of Example 6 within 30 days. FIG. 13D shows the tumor-growing curves of the mouse tumor models of Example 7 within 30 days. FIG. 14 shows the survival rates of the mouse tumor models of Control group 2, Comparison group 2, Example 6 and Example 7 within 30 days. As shown in FIG. 13A to FIG. 13D, there are no tumor suppression effect in the mouse tumor models of Control group 2 and the mouse tumor models of Comparison group 2. However, at least two of the mouse tumor models have the effects of tumor growth delay in both Example 6 and Example 7, and the mouse tumor models of Example 7 have excellent tumor suppression effect compared with the mouse tumor models of Control group 2 and the mouse tumor models of Comparison group 2. As further shown in FIG. 14, the survival rates of the mouse tumor models of Control group 2, Comparison group 2, Example 6 and Example 7 are respectively 0%, 17%, 33% and 100% at Day 30 of the experiment. According to the above, the pharmaceutical composition of the present disclosure can effectively suppress the tumor growth and enhance the survival rate of the cancer treatment. Further, the tumor suppression effect and the survival rate of the cancer treatment can be further enhanced when the pharmaceutical composition of the present disclosure is combined with the chemotherapy drug in use.

### 2. Analysis of the Effect of the Pharmaceutical Composition of the Present Disclosure Used to Stimulate the Secretion of Cytokines

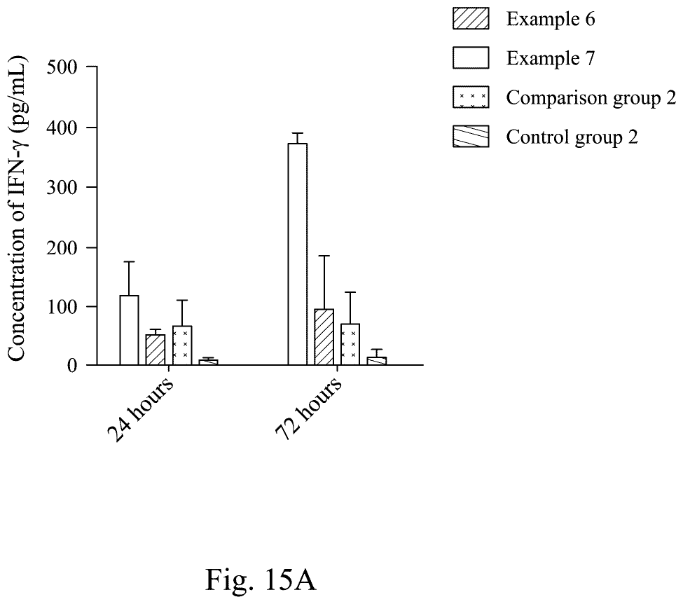

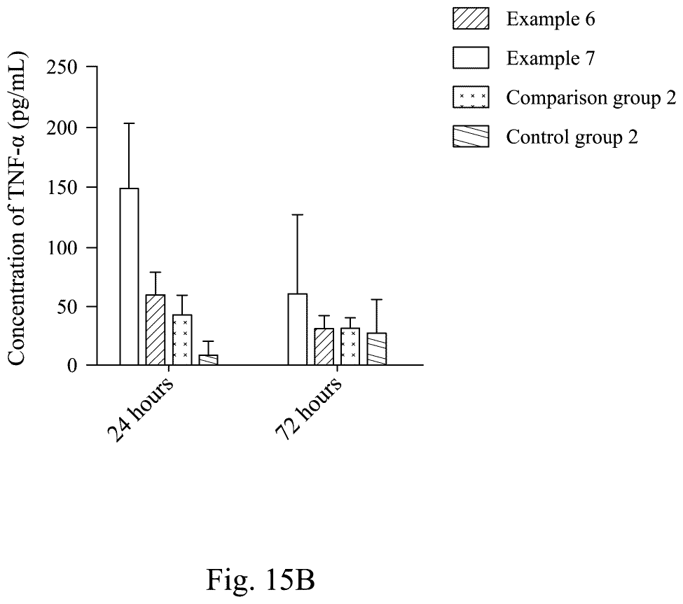

Reference is made to FIG. 15A and FIG. 15B. FIG. 15A shows the results of the concentration of IFN-γ in serum at 24 hours and 72 hours after the mouse tumor models of Control group 2, Comparison group 2, Example 6 and Example 7 are administrated with respective treatment dosages. FIG. 15B shows the results of the concentration of TNF-α in serum at 24 hours and 72 hours after the mouse tumor models of Control group 2, Comparison group 2, Example 6 and Example 7 are administrated with respective treatment dosages. As shown in FIG. 15A and FIG. 15B, the concentrations of IFN-γ and the concentrations of TNF-α in the serums of the mouse tumor models of Example 6 and Example 7 are higher than those of Control group 2 after the mouse tumor models are administrated with respective treatment dosages for 24 hours and 72 hours. The results show that the concentration of IFN-γ and the concentration of TNF-α in the cells can be effectively increased by the pharmaceutical composition of the present disclosure. Further, the concentration of TNF-α in the serum of the mouse tumor models of Example 7 is much more than that of Comparison group 2 after the mouse tumor models are administrated with respective treatment dosages for 24 hours, and the concentration of IFN-γ in the serum of the mouse tumor models of Example 7 is much more than that of Comparison group 2 after the mouse tumor models are administrated with respective treatment dosages for 72 hours. The results show that the IFN-γ and the TNF-α can be effectively expressed from the cells after the pharmaceutical composition of the present disclosure is combined with the chemotherapy drug in use.

### 3. Analysis of the Effect of the Pharmaceutical Composition of the Present Disclosure Used to Activate a T Cell

In order to further analyze the changes of the immune responses of the mouse tumor models of each group, the aforementioned animal experiment is repeated, the mouse tumor models of each group are sacrificed at Day 15 of the experiment, and tumor samples and spleen samples are collected from the mouse tumor models. Next, the tumor samples and the spleen samples are homogenized and immunostained with CD4 antibody, CD8 antibody and CD69 antibody, and the activations of the CD4⁺ T cells and the CD8⁺ T cells in each group are analyzed by the flow cytometry. In the present experiment, a total cell number of each of the tumor samples is 104 cells, and a total cell number of each of the spleen samples is 104 cells.

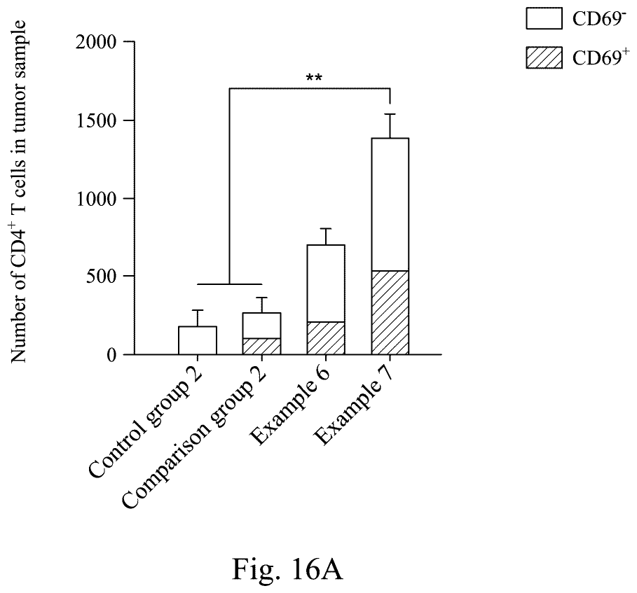

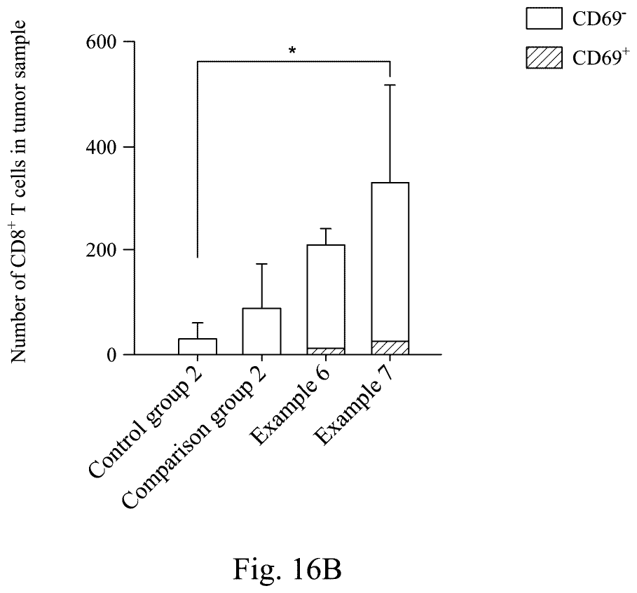

Reference is made to FIG. 16A and FIG. 16B. FIG. 16A shows the quantitative results of CD4⁺ T cells in the tumor samples of the mouse tumor models of Control group 2, Comparison group 2, Example 6 and Example 7 on Day 15 of the experiment. FIG. 16B shows the quantitative results of CD8⁺ T cells in the tumor samples of the mouse tumor models of Control group 2,Comparison group 2, Example 6 and Example 7 on Day 15 of the experiment. Specifically, in FIG. 16A and FIG. 16B, the mark “*” represents a statistical difference in comparison with Example 7 (p<0.05), and the mark “**”represents p<0.01. Further, if the definitions of marks shown in the following drawings are the same as those shown in FIG. 16A and FIG. 16B, those will not be described again.

As shown in FIG. 16A, both of the numbers of CD4⁺ T cells expressing CD69 in the tumor samples of Example 6 and Example 7 are higher than those of Control group 2 and Comparison group 2. As further shown in FIG. 16B, both of the numbers of CD8⁺ T cells expressing CD69 in the tumor samples of Example 6 and Example 7 are higher than those of Control group 2 and Comparison group 2.

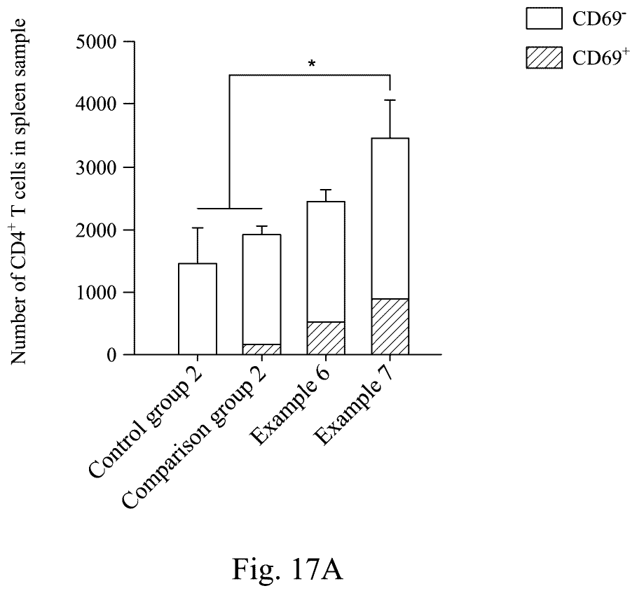

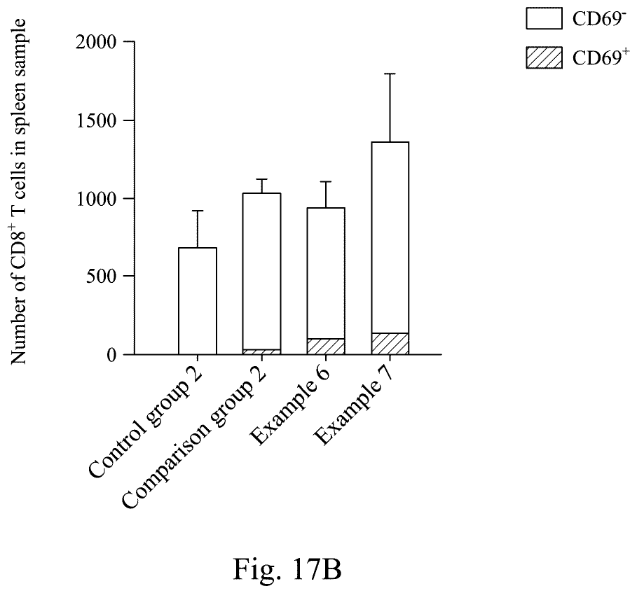

Reference is made to FIG. 17A and FIG. 17B. FIG. 17A shows the quantitative results of CD4⁺ T cells in the spleen samples of the mouse tumor models of Control group 2, Comparison group 2, Example 6 and Example 7 on Day 15 of the experiment. FIG. 17B shows the quantitative results of CD8⁺ T cells in the spleen samples of the mouse tumor models of Control group 2,Comparison group 2, Example 6 and Example 7 on Day 15 of the experiment. As shown in FIG. 17A, both of the numbers of CD4⁺ T cells expressing CD69 in the spleen samples of Example 6 and Example 7 are higher than those of Control group 2 and Comparison group 2. As further shown in FIG. 17B, both of the numbers of CD8⁺ T cells expressing CD69 in the spleen samples of Example 6 and Example 7 are higher than those of Control group 2 and Comparison group 2.

According to the above, the T cells can be effectively activated by the pharmaceutical composition of the present disclosure. More particularly, the CD4⁺ T cell and the CD8⁺ T cell in a tumor and a spleen can be effectively activated by the pharmaceutical composition of the present disclosure so as to promote the immune responses.

### 4. Analysis of the Effect of the Pharmaceutical Composition of the Present Disclosure Used to Reverse the Anti-Inflammation

In the present experiment, the proportions of M2 macrophages in the tumor samples of the mouse tumor models are analyzed by the flow cytometry so as to evaluate the immunosuppressive levels of the mouse tumor models of each group and analyze the effect of the pharmaceutical composition of the present disclosure used to reverse the anti-inflammation.

Further, the M2 macrophage can secrete growth factors and inflammatory factors to suppress the inflammation, and the M2 macrophage can promote the deactivation of the immune responses of the host in the tumor environment, and the proliferation of cancer cells can be enhanced. Therefore, the immunosuppressive level of the mouse tumor models of each group can be evaluated by analyzing the proportions of M2 macrophages in the tumor samples of the mouse tumor models of Control group 2, Comparison group 2, Example 6 and Example 7.

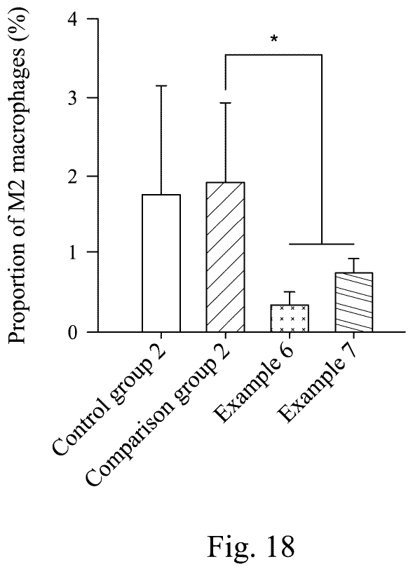

Reference is made to FIG. 18, which shows the results of the proportions of M2 macrophages of the mouse tumor models of Control group 2, Comparison group 2, Example 6 and Example 7 on Day 15 of the experiment. Specifically, in FIG. 18, “the proportion of M2 macrophages” indicated on the vertical axis represents the proportion of macrophages expressing CD206, wherein the CD206 is a M2 macrophage maker. Further, the mark represents a statistical difference in comparison with Comparison group 2 (p<0.05). As shown in FIG. 18, both of the proportions of M2 macrophages of the mouse tumor models of Example 6 and Example 7 are significantly lower than those of Control group 2 and Comparison group 2. The result shows that the immunosuppressive effect of M2 macrophage can be effectively reversed by the pharmaceutical composition of the present disclosure, so that it is favorable for stimulating the immune responses.

As shown in the aforementioned results, the protein-based nanoparticle of the present disclosure is prepared by infecting the producer cell with the recombinant baculoviruses according to the method for preparing protein-based nanoparticle for self-packaging and delivering mRNA of the present disclosure, so that the production efficiency of the protein-based nanoparticle of the present disclosure can be effectively enhanced to 11 times compared with the production efficiency of the nanoparticle prepared by the current method of plasmid transfection, and the production costs, storage costs and shipping costs thereof can be reduced. Further, by the arrangement of the surface of the protein-based nanoparticle having the engineered envelope protein, the transfection efficiency of the protein-based nanoparticle of the present disclosure can be effectively enhanced, and the protein-based nanoparticle can have excellent cell-targeting property, so that the potential of the protein-based nanoparticle of the present disclosure used in treating cancers and other diseases can be enhanced, and the protein-based nanoparticle, the preparation method thereof and the pharmaceutical composition of the present disclosure can have excellent market application potential.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A method for preparing protein-based nanoparticle for self-packaging and delivering mRNA, comprising:
providing a first donor plasmid, a second donor plasmid and a third donor plasmid, wherein the first donor plasmid comprises a nucleocapsid protein gene and an envelope protein gene, the second donor plasmid comprises an engineered envelope protein gene, and the third donor plasmid comprises a target gene;
performing a plasmid transposing step, wherein the first donor plasmid is transposed to a first shuttle vector, the second donor plasmid is transposed to a second shuttle vector, and the third donor plasmid is transposed to a third shuttle vector, wherein the first shuttle vector is transfected to a first virus-amplifying cell, the second shuttle vector is transfected to a second virus-amplifying cell, and the third shuttle vector is transfected to a third virus-amplifying cell;
performing a recombinant virus preparing step, wherein the first virus-amplifying cell, the second virus-amplifying cell and the third virus-amplifying cell are cultured so as to obtain a first recombinant baculovirus, a second recombinant baculovirus and a third recombinant baculovirus, wherein the first recombinant baculovirus is obtained from the first virus-amplifying cell, the second recombinant baculovirus is obtained from the second virus-amplifying cell, and the third recombinant baculovirus is obtained from the third virus-amplifying cell; and
performing a transducing step, wherein the first recombinant baculovirus, the second recombinant baculovirus and the third recombinant baculovirus are used to infect a producer cell so as to express a nucleocapsid protein, an envelope protein, an engineered envelope protein and a target RNA, and the nucleocapsid protein, the envelope protein, the engineered envelope protein and the target RNA are self-assembled so as to form a protein-based nanoparticle, and the protein-based nanoparticle is for self-packaging and delivering mRNA;
wherein the protein-based nanoparticle comprises a lipid carrier, the nucleocapsid protein, the envelope protein, the engineered envelope protein and the target RNA, the target RNA and the nucleocapsid protein are located in the lipid carrier, the target RNA is covered by the nucleocapsid protein, and the envelope protein and the engineered envelope protein are separately located on a surface of the lipid carrier.

2. The method of claim 1, wherein the nucleocapsid protein gene is a PEG10 gene.

3. The method of claim 1, wherein the envelope protein gene is a vesicular stomatitis virus glycoprotein (VSV-G) gene.

4. The method of claim 1, wherein the engineered envelope protein comprises an epidermal growth factor receptor (EGFR) single-chain variable fragment.

5. The method of claim 4, wherein the engineered envelope protein gene has a sequence of SEQ ID NO: 3.

6. The method of claim 1, wherein the target gene comprises an interleukin-12 (IL-12) gene or an OX40L gene.

7. The method of claim 1, wherein the producer cell is a human embryonic kidney cell 293T (HEK 293T).

8. The method of claim 1, wherein a multiplicity of infection (MOI) of the first recombinant baculovirus to infect the producer cell is 62 to 72.

9. The method of claim 1, wherein a multiplicity of infection of the second recombinant baculovirus to infect the producer cell is 28 to 38.

10. The method of claim 1, wherein a multiplicity of infection of the third recombinant baculovirus to infect the producer cell is 45 to 55.

11. A protein-based nanoparticle prepared by the method for preparing protein-based nanoparticle for self-packaging and delivering mRNA of claim 1.

12. A pharmaceutical composition, comprising:
the protein-based nanoparticle of claim 11, wherein the pharmaceutical composition is for treating a cancer.

13. The pharmaceutical composition of claim 12, wherein the pharmaceutical composition is for treating a colon cancer, a brain cancer, a liver cancer or a breast cancer.

14. The pharmaceutical composition of claim 12, wherein the pharmaceutical composition is for promoting an immune response.

15. The pharmaceutical composition of claim 14, wherein the pharmaceutical composition is for activating a T cell.

16. The pharmaceutical composition of claim 15, wherein the pharmaceutical composition is for increasing a concentration of interferon-y (IFN-γ) and a concentration of tumor necrosis factor-a (TNF-α) in a cell.

17. The pharmaceutical composition of claim 16, wherein the T cell is a CD4⁺ T cell or a CD8⁺ T cell.

18. The pharmaceutical composition of claim 12, wherein an effective dose of the target RNA in the protein-based nanoparticle in the pharmaceutical composition is 1×10⁷ copies.
